(19) United States
(12) Patent Application Publication
(10) Pub. No.: US 2009/0125050 A1
(43) Pub. Date: May 14, 2009

(54) Intradermal device introducing method and apparatus

(76) Inventor: Linda Dixon, Kailua, HI (US)

Correspondence Address: SMITH, GAMBRELL & RUSSELL, 1130 CONNECTICUT AVENUE, N.W., SUITE 1130, WASHINGTON, DC, 20036, US

(21) Appl. No.: 12/289,538

(22) Filed: Oct. 29, 2008

Related U.S. Application Data
(60) Provisional application No. 61/000,744, filed on Oct. 29, 2007.

(51) Int. Cl. A61B 17/34

(52) U.S. Cl. 606/186

(57) ABSTRACT

A plurality of different intradermal needle penetration application techniques, preferably both of a powered and non-powered application type, are featured that enable a wide variety of applications with preferably only a single needle device that includes a plurality of needles with preferably a single tip slope edge arranged at a gentle slant, preferably a 30-35° slant, or a plurality of such sloped needle tip edges. The needle arrangement enables the needle set to enter at the same time the skin of the subject to an equal desired depth in consistent fashion. The same needle device can be used for point marking, lining and shading with ease. The application processes include use of a plurality of the provided application techniques preferably in a favored order of application and intermixing the point, lining and/or shading techniques to achieve a highly efficient and rapid application process. Also, a holder tip is provided which preferably has outlet edging that extends or extend parallel with the needle tip line edge(s) of the needle device.

### CROSS REFERENCE TO RELATED APPLICATIONS

This application claims the benefit of U.S. Provisional Application No. 61/000,744, filed Oct. 29, 2007, which application is incorporated herein in its entirety.

### BACKGROUND OF THE INVENTION

For centuries efforts have been made to achieve enhanced coloration of skin for a myriad of reasons. Historically, a variety of implements have been used to indelibly color the skin, ranging from sharply pointed bones, teeth, thorns, guitar strings, safety pins, wood, plastic or any metallic object sufficient to penetrate the skin with color so as to leave a visible mark. These colorful or black and white marks, also known as tattoos, have been applied for adornment, symbolic, patriotic, ethnic or religious identification and artistic expression on a human canvas of skin. Interest also exists in the area of cosmetic, corrective or camouflage tattooing (medical micropigmentation) as a method to provide, for example, permanent makeup, or restore color and/or symmetry to the eyebrows, lips, eyelids, breasts, scars and skin conditions from losses suffered by aging, surgical procedures, birth defects (cleft lip), cancer treatment (alopecia) or skin changes (hypopigmentation—vitiligo).

Tools to implement a desired intradermal coloration of the skin with a penetration implement have also evolved over the years. These include early handtools such as bamboo sticks with strapped needles (a technique still common today in parts of Asia) and motor driven intradermal injection devices. These devices are relied on to inject ink, dye or other marking material (referred hereafter collectively as “ink” for brevity) just under the skin, so that the ink is retained within the skin and the color of the ink injection pattern is visible.

As noted above, color has been implanted into the dermal layer of skin with various implements including coil machines (reciprocating), rotary pen machines and handtools. Provided below is a description of some of the features of such devices as well as some of the use techniques associated with them.

Handtool: Handtools, or the manual method, grip needles in a row at a 45-52° slant, in similar fashion to the manner in which an Xacto® knife grips a blade. The needles are pressed and then rotated upward which generates a “Velcro” ripping sound due to the lifting of the skin by the needles. Skin damage may occur and/or the color may not last as long as that put in with a machine. A lower pigment density or color particles per area of skin results from the handtool or manual technique compared to a machine that is operating with the same needle device for the same period of time. This is because the needles enter the skin many times faster with a machine than when applied by the human hand in a tapping motion.

Rotary Pen and Coil Machines: Needles for these machines, used with power, are configured in various groups with the tips of the needles flush with a flat surface when they contact the surface at a 90° angle or straight down.

When the operator tilts the handpiece the needles, by definition, enter the skin at different levels. The result is uneven or blotchy color or a reduced amount of color implanted into the correct level of the skin, being the dermis. The healed result may only reflect 30-50% of the color inserted at the time of application.

The motorized devices normally comprise a skin-penetrating needle which has the capacity to retain some quantity of ink, a mechanism to reciprocate the needle for repeated punctures of the skin to implant the ink under the skin in the desired pattern, and a housing for the device which the operator holds and often uses to guide the device. There is also typically an off-on switch and power source for the reciprocating drive mechanism. With some devices the operator repeatedly dips the needle into an ink pool to coat the needle, while other devices have built-in reservoirs for the ink from which the ink is fed continuously to the needle.

A number of different devices, particularly with different types of reciprocating needle drives, have been disclosed over the years. Typical of such devices are those described in U.S. Pat. No. 2,840,076 (Robbins: 1976); U.S. Pat. No. 4,508,106 (Angres: 1985); U.S. Pat. No. 4,644,952 (Patips et al.: 1987); U.S. Pat. No. 4,798,582 (Sarath et al.: 1989); and U.S. Pat. No. 5,279,552 (Magnet: 1984).

Examples of pen like (non-motorized) skin marking devices can be seen in U.S. Pat. No. 4,655,912 (Burton: 1987) and U.S. Pat. No. 5,810,862 (Pilamanis: 1998).

Intradermal pigment injection instruments or implements include the use of singular needles. Multi-tip array needles are also featured in the above noted Angres, Pilmanis, and Sarath patents as well as in U.S. Pat. No. 6,030,404 (Lawson et al.: 2000). Needles have historically been made from acupuncture or sewing needles with little thought for needle texture, taper or diameter or intended depth of insertion or color carrying capacity of a needle grouping (the distance between the needle tips). More recently, attention has been paid to needle design to account for penetrating the skin to deliver the ink with the depth being based on insertion depth which, in turn, is dependent upon a variety of factors including the taper and/or diameter of the penetrating needle, the resistance level of the material being penetrated (e.g., the toughness of the skin) as well as environmental factors such as the dryness of the needle and/or skin being penetrated and whether there is wetting or lubrication on the needle or on the skin being penetrated (or other body material) and the location of penetration; e.g. the face or the body.

The age of the person can have an influence on the resistance level to penetration as aging skin tends to lose in collagen level and turgor so as to become more or less resistant to needle penetration and is also more susceptible to tissue damage from any trauma including tattoo needles. One's skin also typically becomes thinner than in one's youth, thus playing a role relative to ink penetration levels. Hormonal effects such as hypothyroidism resulting in thickened, puffy skin and medication as well as ethnicity also play a role in determining skin characteristics.

“Single point” needles are typically relatively larger needles that are designed and used alone relative to the holder for line generation (e.g., single line or areas following extensive multiple line repetition). In view of their size, these single point needles typically are more traumatic on the skin leading to greater puffing, etc., which can make ink application more difficult and less error free (e.g., if puffing and distortion initiates while the ink application is ongoing in the same area). Single point needles do allow, however, for high definition location application particularly in difficult to reach areas or when attempting to set initial external boundary regions.

Multiple needle configurations (hereafter multiple needle arrangements or sets of needles will also be referred to as a “needle” for simplicity although they may comprise a plurality of individual needles in a set; reference will also be made to “needle devices” as another manner of describing single needle or multiple needle configurations) such as that described in Lawson are used to penetrate the skin over large areas. They are, however, not always well suited for areas such as those described above where a certain skin topography, desired ink configuration and/or a body arrangement makes the particular array arrangement of the multi-array needles ill suited for a desired use. This entails having to switch out multiple needle types to accommodate the application requirement or the use of a plurality of different holder/needle set combinations.

### SUMMARY OF THE INVENTION

The present method includes an embodiment that uses a single needle device comprised of a plurality of needles arranged at a gentle slant, preferably 30-35°, which enter and exit a layer of a subject such as the skin of the subject at a rate of, for example, approximately 60-200 strokes/second. A benefit provided with such a technique is that all the needles enter the skin at the same depth at the same time at, for example, a 32° angle. Delivery is preferably to the superficial dermis when used in skin applications. Procedures under embodiments of the invention are estimated to take 30-50% less time than either a respective handtool or regular machine application and more color is retained. There is considered to be less trauma to the skin which results in faster healing. The color implantation of an embodiment of the present invention's method is evenly reflected so the appearance is more pleasing than color which is implanted unevenly by typical methods and results in blotchy or missing color in areas. Embodiments of the invention are suited for use in human subjects as well as in non-humans as in animals (e.g., mammals).

The effectiveness of the present technique is considered to involve the ability to use a single needle device, preferably in conjunction with a receiving tip, which can be used at least preferably 9 and preferably up to 15 different ways relative to a variety of applications carried out. Usually professionals will use a needle device with 1-3 needles to make a line and then use a needle device with 5-14 needles to shade and/or fill in color. However, preferred techniques herein described achieve the desired methods of implanting color with a single needle device. This saves time and money and, more importantly, protects the skin from ineffective and unnecessary trauma and potentially permanent injury due to localized scar formation.

For example, the present invention is preferably inclusive of a method directed toward providing one or more of the following features:
1. The placement of more color in less time with less trauma and pain;
2. The placement of color into the same level consistently for a smooth, even, velvety blanket of color;
3. The avoidance of “blotchy” healed color;
4. The ability to work readily on both curved and flat surfaces;
5. The ability to make brow “hairs” rather than “hairstrokes”; and
6. A technique that replaces discouragement with confidence.

The present invention, among other features, facilitates the proper application of an application material (e.g., ink, dye, pigment or other marking material including fluid and dry based materials with reference being made herein to “ink” as a shorthand representation of the noted various application materials), thus helping to avoid re-applications. Preferred embodiments of the present invention provide the applicator with greater versatility in the application process which provides for efficiencies and a deduction in application time, such as when dealing with the requirement associated with facial cosmetic applications or the like, with the ability to use a single needle device through all application stages of the desired location of application providing for increased efficiency and the lessening of complexity and possible error producing events during the application. Another useful application includes treatment of scars for burn survivors, accident victims and children or adults with facial deformities due to a cleft-lip and other deformity areas.

What the present invention provides is also well suited for placement of color into the curves of skin. That means it takes less time to complete a procedure involving curves in the skin as in lips, brows, thick or smudgy eyeliner and areolas. An embodiment of the method of the present invention features the use of a needle as in a needle set comprised of custom designed needle tips as in those described in U.S. patent application Ser. No. 10/677,115 filed Oct. 2, 2003 (“the '115 application”) which is incorporated herein by reference, although the herein described techniques can be achieved as well with a variety of needle designs (e.g., a single line, sloped edge of 5 commonly configured needles in a set).

Embodiments of the method of the present invention include techniques designed to have the needles slide through the stratum corneum to the superficial dermis and deposit color in a layer like a “blanket of color” which is facilitated under these embodiments of the present invention by using both a special tent angle and depth which provides color into the skin on a consistent basis. Needles and tips, that are examples of components well suited for carrying out the method of the present invention, are available for the Infiniti®, Freedom and other pen machines that take a 2″ needle. Handpieces that have an angle such as the KP96, Midas or traditional coil machines aren't as efficient as they require extra care since the handle design can hit the top of your hand while working with the angled needles, but are not excluded under embodiments of the invention. The invention can also be used for other medical, non-skin use such as medical pigment application to non-skin areas like the cornea of an eye, although the preferred usage and many of the parameters set forth herein are directed at skin penetration.

The method of the present invention is workable with a digital application machine with the machine preferably having a handpiece that takes “special cartridges”. In the field black, white and gray represent color coded cartridges used for various machines. Special cartridges are available for the MTDerm digital handpiece. Moreover the preferred color application methodology of the present invention is highly versatile for use in a variety of applications such as lips, breasts, camouflage, eyebrows, medium to thick eyeliner, smudgy eyeliner, and eyeshadow as a few examples. The benefits of the present invention relative to a standard approach are better suited for the former applications usages of lips, breasts, camouflage, eyebrows, fine to thick eyeliner, eyelash enhancement and smudgy eyeliner and eyeshadow.

Thus, a preferred embodiment of the present invention provides a single needle device that is well suited for use in a wide variety of application techniques (e.g., 9 to 15 is illustrative) and also in a wide number of application settings, with a high level of intradermal color implantation performance (e.g., a high color retention efficiency) achievable in those application settings.

Embodiments of the present invention are also designed to facilitate achieving novel color patterns as an operator can make patterns or footprints of color in the skin using the methodology of the present application such as utilizing a slanted needle in a machine to achieve color patterns, that are considered not achievable to the same degree alone by either the handtool or a machine needle configuration before (e.g., the windshield wiper stroke and graduated hairstroke described below).

There can also be achieved under the present invention, a multiplicity of color patterns with a single needle device. That is, in addition to even color placement at the same depth at the same time, the methodology of an embodiment of the present invention facilitates achieving, with the novel use of a slanted needle in a machine, nine different patterns of color. Ordinary needle configurations in either a handtool or machine achieve at best two different patterns of color in the skin.

The present invention also helps avoid the tendency of ordinary needles, which are being used in a machine, to deposit color at different levels resulting in a blotchy healed color in the skin. The techniques under the present invention also include providing for depositing color at the same depth at the same time and, most preferably, at a 32° angle. This combination results in unprecedented color retention.

The results of the present invention are highly beneficial in that there is a remarkable visible difference relative to traditional methods of tattooing and cosmetic tattooing in the healed result. There is less color loss during the healing period. Historically, professionals were accustomed to losing 30-50% of the color they placed into the skin during the healing process. This can be accounted for because it is considered the only 50-70% of the color was being placed at the correct depth in the dermis under the conventional techniques. If the color is placed too deep then there is bleeding, swelling and color loss. What color is left at that depth is not easily visualized because of the depth that light travels into the skin and poor reflection. Secondly, color that is placed too shallowly is lost during the healing process similarly to skin peeling after a sunburn as well as normal exfoliation. For instance, there is considered to be a less than 20-30% loss in color between application and the healed result under preferred embodiments of the present invention. Also, there is considered to be a greater than 70% -80% placement of color at a desired depth relative to the application desired under preferred embodiments of the present invention. Hence, the skin does not have to be “overworked” and damaged during the coloring process with embodiment of the present invention as the color is implanted at the correct depth more uniformly and effectively than historically considered achievable.

The needle devices and techniques of preferred embodiments of the present invention are also applicable for use in scars, including facelift scars, hair transplant scars, mastectomy scars, cleft lip and scars resulting from trauma of any kind. The needles are also helpful for temporary treatment of some facial wrinkles, both active and passive. The technique has been used with positive results to camouflage the skin and for acne scars. While color application is possible, no color is typically involved in these cases and the needles in the aforementioned sloped configuration are most commonly moved in a side-to-side stroke pattern as described in greater detail below. Further, while dry needling of the skin has been done in the past, it is considered to have been with a three point needle which is considered not suited for obtaining an even depth of penetration upon the anticipated deviations from a 90° application, as needle tips penetrate to different skin depths as soon as tilted.

An additional advantage of an embodiment of the present invention is one can use a single handpiece such as that associated with a powerizable tool system (e.g., a digital application machine with a single needle device (e.g., a 5 needle common line tip edge)) in both a powered mode and a non-powered mode relative to the techniques described herein. This further provides for continuing an application process even when power goes out. Furthermore, having, for example, 3 to 5 (or more) needles in a preferred gentle slope needle set arrangement under the present invention can also be advantageous in that any single needle point step or any step with less than all tips inserted (e.g., a graduated line technique) utilized in a desired application procedure can be more closely controlled as the remaining needles in the tapered line edge set provide for a visualization of a desired stop point (the second or third or some other number needle in the tapered needle set relative to skin surface that is not to be penetrated by that needle). Thus, the present invention can avoid the situation where a single needle is repeatedly inserted and removed with no knowledge or visualization of the depth each time.

The color material relied upon to achieve the desired color under the present invention can be any of those on the market that preferably do not contain water or excessive amounts of alcohol as such pigments can dry out and clog the needles. The use of Kolorsource™ color material, with Kolorsource™ lip colors is considered to be a high quality color material which high quality is even more apparent when such material is implemented in accordance with an application technique featured in the present invention.

To facilitate the below needle device application discussion under the present invention, reference is made to the following needle point level categories of “A” (needle in superficial papillary dermis); “B” (needle in mid-dermis pigment in dermal layer of skin), and “C” (needle in deep “reticular” dermis). The correspondingly labeled illustrations found further in the present application provide an illustration of these positions.

To facilitate the discussion below a description is provided of needle “footprints”. A footprint of a needle is the pattern or trail of color made in the skin by that needle. A 3-flat has a footprint that is either 3 wide (like a rake or 1 wide if used with the needles behind each other). A 9-magnum can be used 2-wide or up to 5-wide and is typically two rows thick. A 3-point round needles is only ever 2 needles wide. The correspondingly labeled “footprint” illustrations found further in the present application provide added illustrations of the same.

The following characteristics of a needle are also provided to facilitate the discussion herein.
1. Number of needles in the needle device;
2. Configuration;
   a) Flat
   b) Round
   c) Magnum
   d) Slope
3. Taper of Needle;
4. Texture of Needle;
5. Needle Size (diameter); and
6. Taper of Needle: tip geometry.

An additional factor in intradermal color application methods is the color density (e.g. the density of the pigment in a solution or mixture of pigment and liquid). For example if the density is too weak (e.g., too much water added relative to the amount of pigment) the benefits of a consistent and proper level application can be degraded due to the density of the color. Pigment density can have a role in the healed color and how long the color will last. Further under preferred application techniques of the present invention there is achieved a placement of the color in the superficial dermis for desired applications as that placement acts to “blanket” other pigments in our skin that exist naturally. Melanin is in the epidermis which overlies the dermal location of implanted color so it is acts as a color filter through which one observes the color deposited during tattooing. Therefore, in individuals with high-melanin content, such as dark-skinned or black persons, the color in the dermis is not as easily visible. However, being able to place color in the superficial dermis at the same level in this “blanket” effect offers improved results from corrective application over older undesirable color placed in deeper layers of the dermis.

Examples of methods for intra-dermal color technique using the techniques of embodiments of the invention are described herein and preferably involve a single needle device comprising a multiple needle set that is usable in a wide variety of applications and can be used in a wide variety-of application techniques to achieve the desired results.

For the below listed applications (e.g., Lips) there is provided an example of the method of application of the needle device (e.g., a single needle device with preferably 3 to 5 or more (“more” including, for example, a needle device with 6 to 12 needles in a set in one or more tip line or edge planes) for the given application). In the examples below, a single needle device having a 5 needle set, single edge, sloped tip plane is used. It is preferable to use at least 3 needles within the needle device and preferably along a common tip edge line as some benefit is lost when going below 3 needles in the unique hybrid integration featuring an integration of a needle application of single or <3 point tip insertion for certain application steps and 3 or more tip insertions for other application steps. An example of a greater than 5 needle approach can be seen in the discussion below of having a tip formed to accommodate a two sloped edge needle device having a common vertex needle tip (or series of tips in a horizontal edging line (intermediate vertex region)) plus two lines or edges extending there out from the vertex point or vertex region with one sloped edge line having, for example, two needles out from a common vertex needle (or the closest needle of a vertex region) and the other sloped edge having, for example, four needles going out from that common vertex needle (or closest needle of a vertex region).

The advantage of using sloped needles in a preferred embodiment of the invention is that they can line, shade, fill and make the finest hair strokes all in one needle (available in a 3-slope and a five-slope). All needles can be placed at relatively the same depth at the same time, leaving an even, velvety layer of color at the same (and desired) depth. Examples of types of movement featured under preferred application techniques of the present invention include the following:

Loop shading—wide back and forth looping motion (or “obovoids”) (preferably not tight circles or an applicator can mistakenly, in essence, drill a hole in the skin.);

Wide—Windshield Wiper Pattern shading, which application techniques is especially good for curved surfaces such as lips (e.g., it is well suited for Cupid's bow and/or Smudgy Eyeliner application;

Lining—preferably uses all needles to make a line in the skin. Can make even or uniform line thickness or graduated line thickness (e.g., hairstrokes);

Single needle or “ballerina”—uses a single needle to make a fine line or etch in area (technique uses highest needle alone).

As some example of some intradermal applications:

Lips—six stroke techniques (in the noted order or in any different sequence involving steps 1 to 6 below):
1. Liner;
2. Windshield wiper to fill cupid's bow on upper lip at midline;
3. Ballerina single needle used to do fine lining and finishing of corners of lips and cupids' bow;
4. Side-to-Side for shading and filling in color;
5. Obovoids or circles for filling in color; and
6. Tapping with machine on for wide lipline.

Eyes (in the noted order or in any different sequence involving steps 1 to 5 below):
1. Tapping for upper or lower eyeliner;
2. Side-to-side for upper eyelid preferably with outer ⅓ to “flare” and make eyes appear larger;
3. Windshield wiper for upper eyeliner “smudgy” effect;
4. Liner for narrower line that tapers toward nose; and
5. Single needle, Ballerina, for very fine dotting or lining, especially toward inside eye toward nose.

Brows (in the noted order or in any different sequence involving steps 1 to 6 below):
1. Graduated hairstroke: Line is thicker and become thinner like a real hair as in a calligraphy;
2. Sweep needles up full width makes 5 hairstrokes at one time;
3. Shading with Back-and-forth, Side-to-Side Stroke; and
4. Line: The needles are preferably rotated 45° from wide to narrow toward the end of the eyebrow known as the “tail”. At full rotation the needles form a single line like ducks in a row.

The present invention also provides for the potential for greater accuracy in application through a high level of instrument adaptation potential for the application areas. This feature is particularly helpful when dealing with cosmetic applications such as eyeliners where the application areas include a wide variety of skin topography and condition changes as well as environment obstructions and avoidance areas (e.g., the eye). Also, the present invention also includes preferred embodiments that lessen the trauma and associated swelling both during and after a procedure so as to facilitate the proper application (e.g. accuracy in both location and depth) of the ink. Through the use of a lubricant such as petroleum jelly applied in conjunction with the potential varied configurations relative to a needle device, there is further facilitated rapid and proper intradermal ink application in even difficult to apply regions. In providing these features under preferred embodiments of the present invention, there is also lessened the need for repeat applications.

An embodiment of the present invention features a needle device with a single row of needles (e.g., single row in a common vertical reference plane) as well as more than one row of needle sets (e.g., two or three parallel, vertical plane rows of needles), with each row having a group of needles with tips arranged to form a sloped line edge (e.g., a single edge or multiple edges as described in the aforementioned U.S. patent application Ser. No. 10/677,115 filed Oct. 2, 2003). Preferably all tips within a common edge present a common plane edge line set for simultaneous contact with a layer of a subject as in the skin of a human. A common row of a two edge needle set spaced from another set of the same is a further example of an embodiment. An embodiment of the present invention also comprises two spaced apart rows of 5 needles that are slanted with the rows being parallel to each other. Additional embodiments include the 3 to 5 needles of a set on a common plane that are of the same individual needle configuration or can include individual needles of a different caliber within that set.

The present invention also features a holder tip which is configured to receive the parallel sets of needle groups (having, for example, one or more contact edges within each needle group and having common or different individual needle characteristics within each group). This embodiment of a holder tip preferably has peripheral free edges defining an outlet opening for the outer extremities of the needles, which free edges or rims extend parallel with the parallel rows of needle edge tips in each needle set, and with those needles preferably extending out a common distance form each respective sloped free edge of the holder tip (e.g., the free edges of the holder tip preferably run parallel with the respective edges defined by the peaks of the needles within each needle group).

### BRIEF DESCRIPTION OF THE DRAWINGS

FIG. 1 illustrates a method technique with single point of a multi-needle device inserted within the skin and the remaining non-inserted tips positioned for rotation in “ballerina” fashion as well as the foot print associated with such a technique (single dot application and lining following a series of ballerina rotations).

FIG. 2 illustrates a method technique with single point of a multi-needle device inserted within the skin and the remaining non-inserted tips not inserted with a non-motorized application mode as in machine off (power off), and/or handtool as well as the foot print associated with such a technique.

FIG. 3 illustrates a method technique with multiple-points (e.g., all) of a multi-needle device inserted within the skin at an angle relative to the surface of the skin that corresponds with the tilt angle of the needle set (all angled the same) to present the tips at a common level below the outer exposed surface of the skin with a motorized application machine off (power off and/or hand-tool) as well as the footprint associated with such a technique.

FIG. 4 shows a technique similar to FIG. 3 but for the application being a “lining” technique involving movement in the arrow direction shown (preferably with the power on);

FIG. 5 shows a technique similar to FIG. 4 but for the graduating of the line by insertion of only some of the needle tips (more than a single point application but less than all tip insertion) and with some of the tips going to a different level of insertion (e.g., a graduated hairstroke line).

FIG. 6 shows a technique with formation of a plurality of lines in parallel fashion based on back and forth (or side to side) shading movement of the needle device.

FIG. 7 shows a windshield wiper stroke involving a rotation with displacement type effect paced on the set of needle tips insertions from a central region to opposite sides.

FIG. 8 shows a dotting or tapping function with reciprocated movement along the axis of the needles in the needle device.

FIG. 9 shows a loop of obovoid sequence of movement in a needle device having all tips inserted during the process.

FIG. 10 shows a needle cartridge with a clear holder tip having a sloped free edge out from which the needles of the needle device can extend a common distance and parallel to the needle tip line plane formed by the tips of the needles in the needle device as well as a support connector (e.g., handheld and/or powered tool connection support).

FIG. 11 shows a needle cartridge featuring a hybrid connector end for connection with, for example, a handheld and/or powered tool as in a digital machine for powered needle reciprocation or skin penetration movement.

FIG. 12 shows an example of a holder tip designed for use with a single lip tip plane with there further being shown a schematic redesign illustration designed to provide for a two line tip plane or two-edge needle device shown positioned above the holder tip.

FIG. 13 shows the needle set of FIG. 12 superimposed upon a single sloped holder tip.

FIG. 14A shows, in perspective, a holder tip suited for a multi-slant edge needle set (two slope accommodation shown)

FIG. 14B shows, in perspective, a holder tip suited for a single-slant edge needle set.

FIG. 15 shows a side elevational view of the holder tip shown in FIG. 14A relative to a horizontal plane providing a frame of reference for slope angle depiction.

FIG. 16 shows an end view of the distal end of the holder tip shown in FIG. 14A.

FIG. 17 shows a side elevational view of the holder tip shown in FIG. 14A.

FIG. 18 shows an additional perspective view of the holder tip of FIG. 14A from a different angle.

FIG. 19 shows a front elevational view of the holder tip of FIG. 14A.

FIG. 20 shows a plan view looking into the proximal or handpiece connector end of the holder tip.

FIG. 21 shows needle sets suited for use under the present invention having three, four and five needle single edge slopes, respectively.

FIG. 22 shows an ordinary flat needle inserted into the skin such that the tips are at different levels and such that there is presented a resultant unevenness in color application.

FIG. 23 shows an example of a 5-round needle with a 90 degree orientation such that there is a resultant five dots of color at an equal depth upon application.

FIG. 24 shows an example of the same needle of FIG. 23 with a tilt relative to the presented surface of the skin such that results in a change in needle tip depth amongst the 5 needle set and a blotchy color pattern.

FIG. 25 shows a single line edge set of needles having a preferred slope angle and shown housed in a holder tip and extending into the skin such that the needle tips reach a common depth and the holder tip free edge is above the skin's exterior surface (shown planar here).

FIG. 26 shows views of two and three line sloped edge sets of needles as described in the '115 application with common vertex point or region.

FIG. 27 shows an example of a uniform line application technique featuring all needles touching the skin at the same depth and same time with a five needle set being illustrated.

FIG. 28 shows a schematic example of a graduated line formation technique (either straight line or flared at end) well suited for eyebrow formation and using the same five needle set as in FIG. 27.

FIG. 29 shows an example of the graduated line technique of FIG. 28 on a skin presentation surface.

FIG. 30 shows a schematic view of the same needle set utilized in FIGS. 28 in a “ballerina” technique featuring a single needle skin penetration amongst the five needle set.

FIG. 31 shows an example of the ballerina technique of FIG. 30 on a skin surface presentation and with the clear holder tip visible.

FIG. 32A shows a schematic presentation of the windshield wiper stroke or technique (a shading technique) showing the needle set and pattern produced when the wrist is held stationary at the center of the stoke and the hand is rotated to produce the illustrated pattern.

FIG. 32B shows the windshield wiper technique in an eyeliner training or practice setting.

FIG. 32C shows the initial positioning of the needle for windshield wiper application.

FIG. 33A shows a schematic presentation of the looping or obovoids formation motion (loose circle) stroke or technique showing the same needle set as described above and a schematic of the pattern produced (an additional shading technique).

FIG. 33B shows the loop technique in an eyeliner training or practice setting.

FIG. 34 shows the loop technique in process relative to a skin presentation surface.

FIG. 35 shows a side to side shading technique (a further shading technique).

FIG. 36 shows the side to side or back and forth shading technique in FIG. 35 in process.

FIG. 37 shows a preferred stroke choice schedule for lip application.

FIG. 38 shows a motorized intradermal injection device.

FIG. 39 shows a zoned skin depth face chart.

FIG. 40 shows a cross sectional view showing a needle in superficial papillary dermis.

FIG. 41 shows a cross sectional view showing a needle in mid-dermis.

FIG. 42 shows a cross sectional view showing a needle in deep “reticular” dermis.

FIG. 43 shows a pigment outline for human skin.

FIG. 44 shows a density comparison for pigment application via a needle approach.

FIG. 45 shows a first set of needle footprint presentation.

FIG. 46 shows a second set of needle footprint presentation.

FIG. 47 shows a comparison illustration of a 5 flat needle set application in a transverse application and in an tilted application and the different depth result for such a tilt.

FIG. 48 shows a preferred 32 degree slope (or about 32 degrees as in plus or minus 5 degrees and more preferably plus or minus 1 to 2 degrees from a true 32 angle).

FIG. 49 shows various footprints provided by a preferred sloped needle with a showing of the needle tips reaching a common depth in the skin when arranged with needle tips edge line parallel with skin surface, which footprints showing dots in a row or lines or rainbow prints.

FIG. 50 shows a lip lining training or practice example.

FIG. 51 shows a first view of a single slope edge, 5 needle set received in a holder tip having a parallel pair of rim edges to the slope edge of the needle.

FIG. 52 shows a similar view of that which is shown in FIG. 51 but at a different angle.

FIG. 53 shows the ballerina technique training or practice example wherein there is used the highest needle alone such that only one needle is brought in contact with the skin during this technique.

FIG. 54 shows a lip lining training or practice example.

FIG. 55 to 58 shows first to fourth brow direction application examples, respectively.

FIG. 59 shows an additional view of a resultant brow application example.

FIG. 60 shows a brow hairstroke training or practice example depiction.

FIG. 61 shows a view of a needle angle application for an eyelid application (e.g., placing the needle tip edge line generally parallel with the underlying skin surface).

FIGS. 62 and 63 show before and after depiction of the use of the present needle technique relative to removing scarring in the region of the areola with a technique inclusive of a shading technique

FIG. 64 shows a compilation set of 9 different application techniques and relative footprints left as well as an indication of preference for non power settings for some of those techniques.

FIG. 65 shows a 5 set of application techniques or stroke options for in eye liner applications.

FIG. 66A shows a preferred lip application sequence under an embodiment of the present invention.

FIG. 66B to FIG. 66E provide an additional breakdown of the application sequence outlined in FIG. 66A.

### DETAILED DESCRIPTION OF THE DRAWINGS

Provided below are illustrations and text to help explain the method of the present invention and associated apparatus which includes a discussion of the different application method steps which provides for the preferred use of a single needle device (includes single plane and single slope edge or multi-slope edge needle sets as well as multi-plane parallel spaced apart needle rows of a single slope edge or multiple slope edge in a particular plane) having a plurality of needles which are arranged for use in below described application steps involving single point insertion, multipoint insertion (some or all) and a variety of movement techniques relative to the needle device and with respect to non-powerized modes and powerized modes.

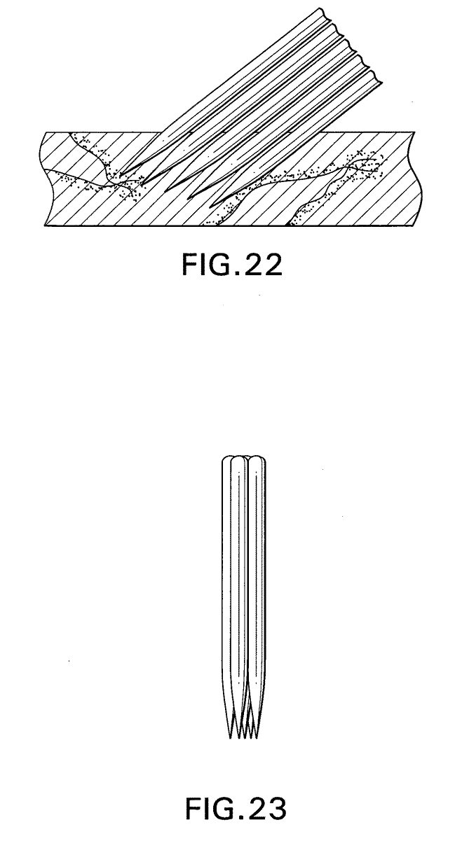

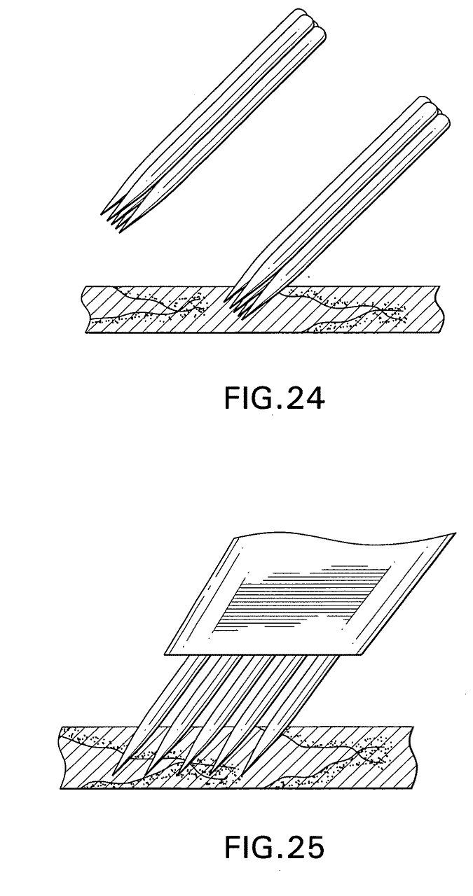

To better appreciate the features of the subject matter of the present invention reference is made to FIGS. 22 to 24 with FIG. 22 showing an ordinary flat needle inserted into the skin such that the tips are at different levels and such that there can result unevenness in color application. FIG. 23 shows an example of a 5-round needle with a 90 degree orientation such that there is a resultant five dots of color at an equal depth upon insertion. However, as seen from FIG. 24, as soon as there is a deviation from a transverse application into the skin there results needle tips extending to a variety of different depths and hence unevenness in the colorization. Further, FIG. 47 shows a comparison illustration of a 5 flat, single edged needle set application in a transverse application and in a tilted application and the different depth result for such a tilt. Also, there can be appreciated that applying the transverse application in a contoured or curved skin setting can result in uneven application as well as the more centered needles penetrating to different depths relative to the more exterior needles if those exterior needles enter at all.

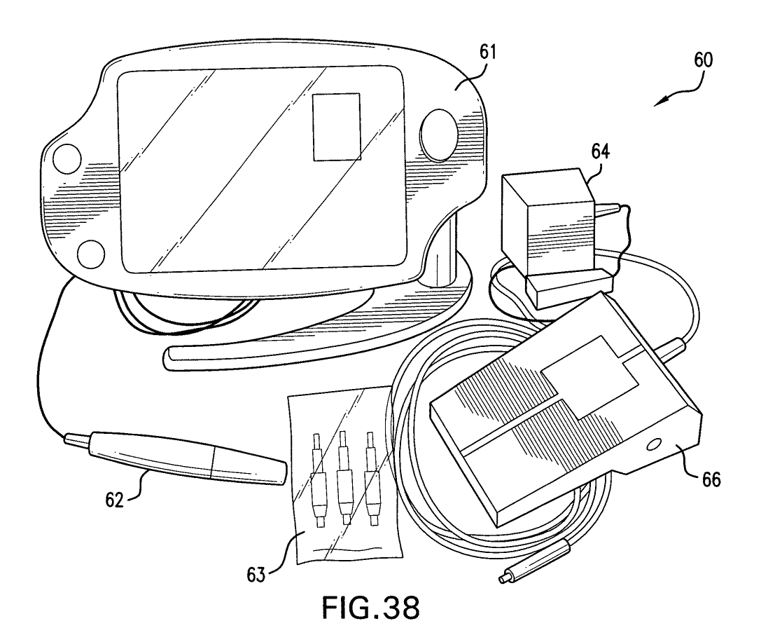

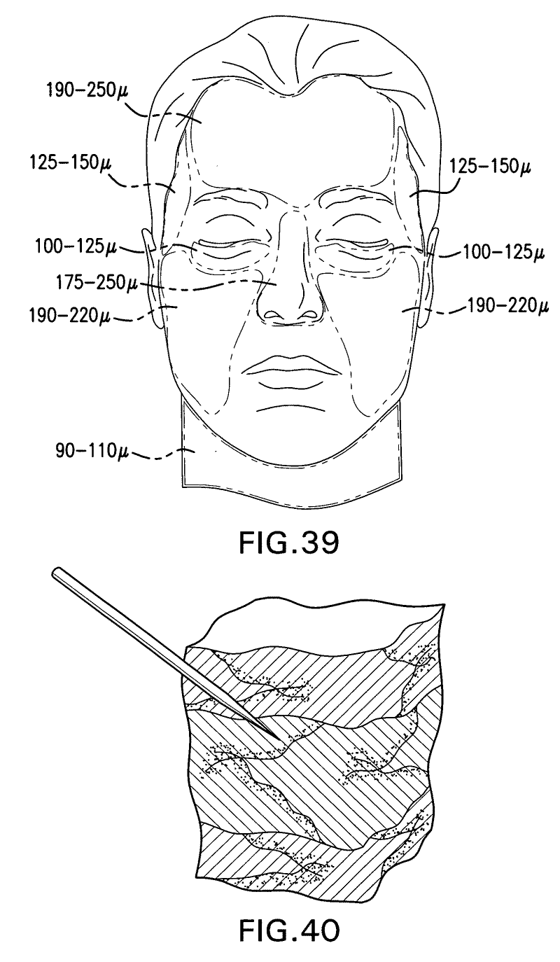

FIG. 39 shows examples of skin thickness ranges in different face regions (which different zones of skin depth ranges can have applications in other regions of the body which, while perhaps, not so pronounced can present different zones of skin thickness in a common region as in the breast area for example). As shown in FIG. 39, there is quite a variety of depth differences and hence having needle insertions that are variable can be problematic as in providing quality colorization which avoids undesired smudging and unevenness as well as poor heal times due to improper application.

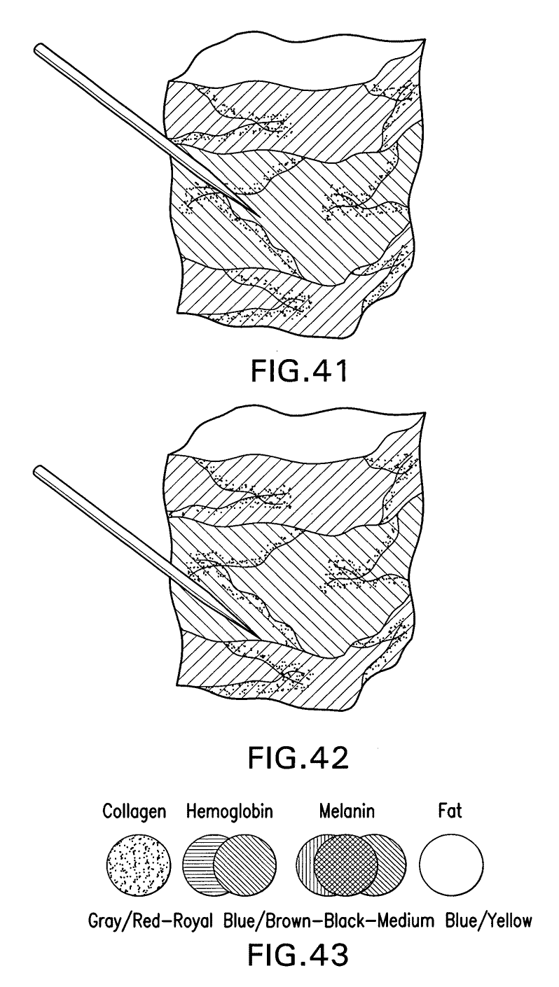

FIGS. 40 to 42 are illustrative of the relative skin and needle depth characteristics with a higher, intermediate and deeper depth shown respectively. That is, in FIGS. 40 to 42 there is shown the needle tip penetration into the skin and achieving a superficial papillary dermis level in FIG. 40, a mid-dermis level in FIG. 42 and a deep “reticular” dermis level in FIG. 43. For example, under preferred application techniques coloring is achieved by a placement of the color in the superficial dermis for desired applications as that placement acts to “blanket” other pigments in our skin that exist naturally. In certain settings, it may be more desirable to penetrate into the mid or deep levels generally illustrated in FIGS. 42 and 42. The superdermis layer is deemed a preferred embodiment for many uses as, for example, melanin is in the epidermis which overlies the dermal location of implanted color so it is acts as a color filter through which one observes the color deposited during tattooing. Therefore, in individuals with high-melanin content, such as dark-skinned or black persons, the color in the dermis is not as easily visible. However, being able to place color in the superficial dermis at the same level in this “blanket” effect offers improved results from corrective application over older undesirable color placed in deeper layers of the dermis.

As an additional illustration of some of the variables that can exist in pigment application via an implantation process, there is shown in FIG. 43 the fact that different pigments in the skin can also have an influence in the overall color following the application of pigment. For example, as shown in FIG. 43 the collagen in the skin can generate a greenish gray pigment color, while hemoglobin can present a blend of shades or hues of blue, red and orange, melanin can present a variety of pigment colors as in brown-black-blue-orange while fat can present, for example, a yellow pigment shade. Care must thus be taken as to avoiding undesired pigment insertions relative to colorization pre-existing in the skin. Providing pigment to varying levels also can lead to undesired and unexpected coloring consequences relative to the resultant product of pigment implantation both in general terms and relative to the different pigment areas in the skin itself.

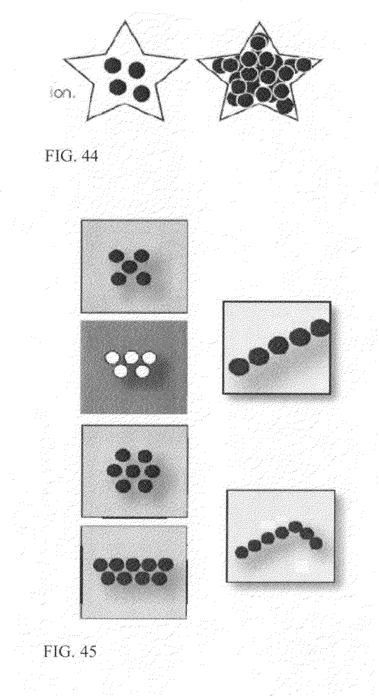

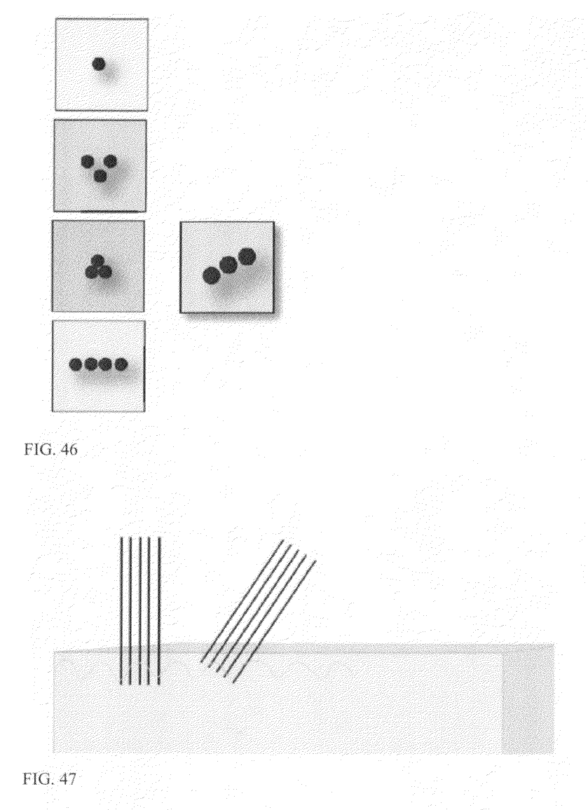

An additional variant in how a resultant process ends up can be seen in the density of needle application (both from a two dimensional level as seen by FIG. 44) as well as a three dimensional standpoint as when there is lacking uniformity in needle tip set penetration. FIGS. 45 and 46 further illustrate that the needle footprint plays a role in how the ink will be applied (e.g., density and application configuration).

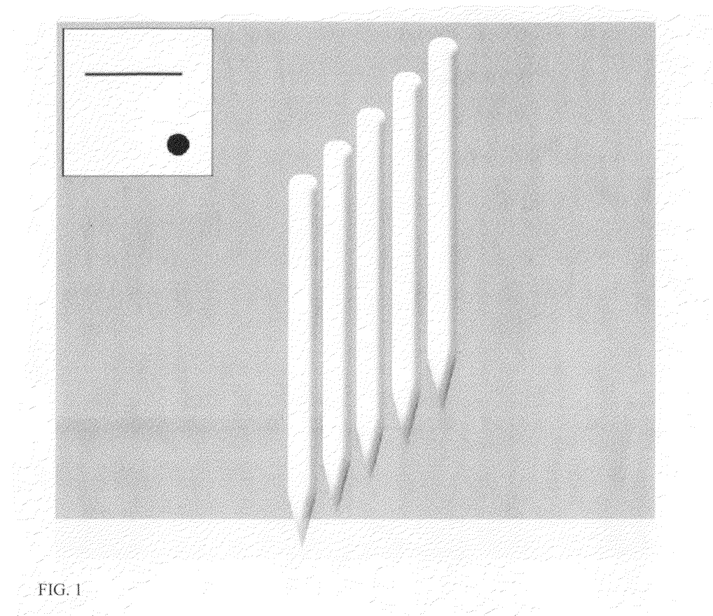

The inventive subject matter provides for the use of one needle device configuration that has a gentle slope (e.g., about 32 degrees) for carrying out a variety of application techniques as in lining, shading, filling, making fine hairstrokes. In a preferred embodiment, a 3, 4 or 5 needle single line slope or those needle designs outlined in the above noted '115 application to the same inventor (application Ser. No. 10/677,115 filed Oct. 2, 2003, which is incorporated by reference) can also be utilized for the preferred techniques described herein. In preferred techniques all needles go to the same depth at the same time, leaving an even, velvety layer of color at the same (and desired) depth (e.g., see FIGS. 40 to 42 discussed above as depth examples). Further the same needle can be used for shading (as side-to-side, windshield wiper and moving loop shading techniques), lining and single point applications. In this regard, reference is made to FIG. 1 which illustrates a method technique with single point of a multi-needle device inserted within the skin and the remaining non-inserted tips being positioned above the region of the skin encompassing the insertion point as to enable rotation of the needle set in “ballerina” fashion as well as the foot print associated with such a technique as in a single point application with one entry location and lining when the “toe” needle is moved along a line pattern. Under the FIG. 1 “ballerina” technique there can be carried out a powered (see the discussion below concerning the powered intradermal injection kit used in needle reciprocation shown in FIG. 38), technique wherein, with power on, the operator turns the handlepiece 180 degrees so that only the topmost needle will contact skin such that in essence there is utilized the “toe” of the needle as the sole needle applied into the skin in “ballerina” fashion. This single needle application is well suited for etching in fine areas in areas such as the Cupid's bow where only a small amount of color is desired. An additional suited application location for the ballerina technique is at the corners of the mouth as well as on the eyebrows for a very fine hairstroke at the beginning of the brow. Care must be taken with the preferred powered ballerina technique as to avoid jabbing the skin accidently, particularly in view of the rotation aspect. Furthermore the slope edge of about 32° (which is preferred for use in the present invention) facilitates having the toe needle tip extend to a desired depth during application and is sufficient for the rotation clearance. A typical handtool has a less gentler slope with a 42 to 52 slope being common for such handtool needle sets.

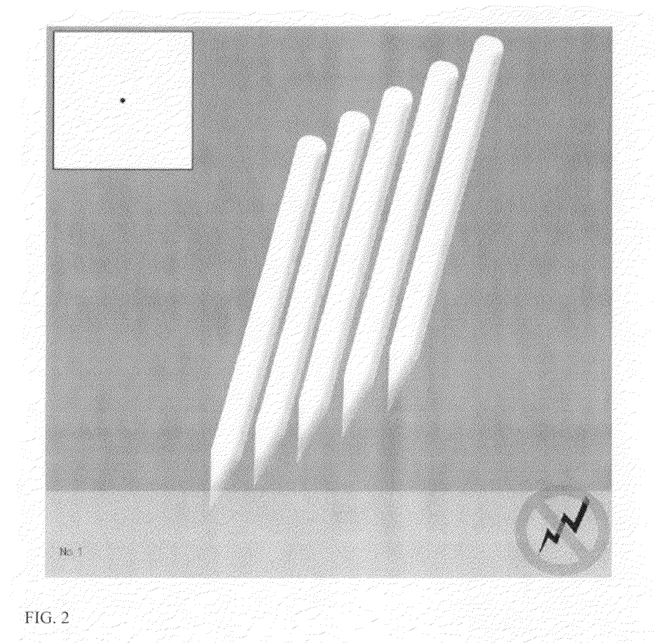

Further, FIG. 2 illustrates a “mark” the skin technique as in one where there is no power and the “toe” needle mentioned above for FIG. 1 is utilized (shown with a slight hand application tilt in FIG. 2). Under the FIG. 2 technique, the toe needle is applied (solely) to the skin to provide a color dot marking. With reference to FIG. 51 there can be seen that the “toe” needle is labeled needle “5” amongst the 5 needle single slope edge set shown in FIGS. 51 and 52 for ease of reference. This FIG. 2 marking technique is well suited for use in lipline applications wherein, for example, the toe needle is used to dot little spots of pigment along the lipline. In this way the FIG. 2 marking technique can be used to provide for precision marking as in marking to provide guidance in a later application step or technique type.

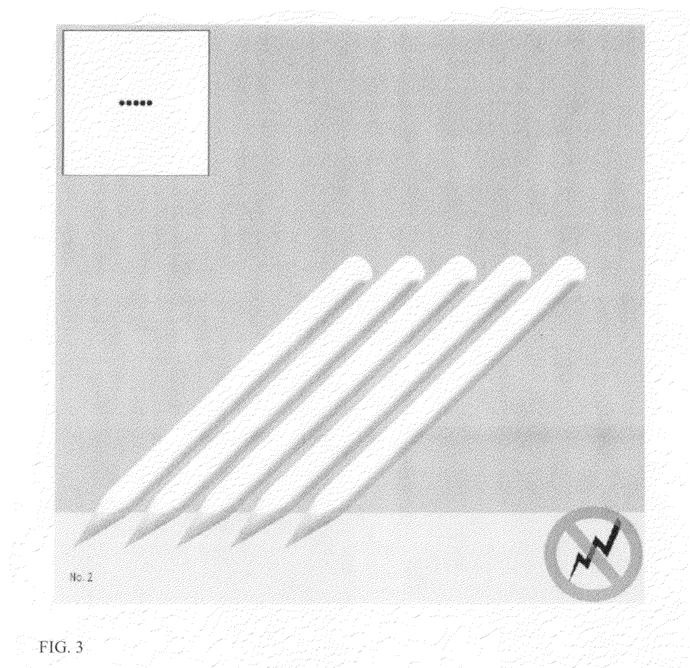

FIG. 3 is representative of a preferably non-powerized, manual technique embodiment wherein again, preferably without power, a user taps the skin in the manner illustrated with or without color as desired. The user should preferably provide for under this technique an arrangement wherein the tips extend the maximum distance out from the tip of the handpiece. This technique is well suited for use when a careful and cautious application is warranted due, for example, the type of area being worked upon. This technique can be used with the same handpiece used for power applications but with the power off and with that handpiece manipulated by hand to tap in color in handtool fashion. This technique can be useful as in, for example, tapping in color when starting an application to the upper eyeliner at the outermost eyelash. For example, a tapping in at a 45 degree angle from the eyelid upward and outward. Once this is completed there can be further carried out in this area a powered application moving inward toward the nose. There can then be rotated the needles so they make a gradual transition to a single line which tapers nicely toward the inner eye. As seen from the discussion herein, there is provided in a preferred embodiment a coordinated power and no-power application approach in relationship to the different techniques in an effort to provide an overall increase in efficiency in the desired project being worked upon. In other words, in a preferred embodiment of some technique sequences under the present invention a sequence can involve non-powered applications preferably with a common needle set holding means (e.g., the below described cartridges and handpiece combinations).

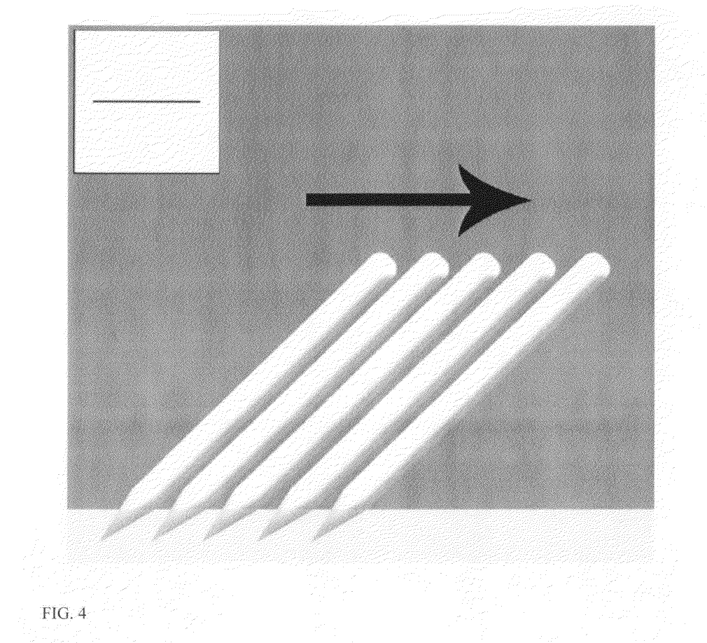

FIG. 4 shows a lining technique which preferably involves a powered mode setting and wherein all needles of the set are inserted into the skin at the same depth at the same time moving to provide a relatively uniform line width. Note also that the needle to the opposite side of the toe needle (or needle “1” of FIG. 51) is leading the way relative to the direction of motion represented by the directional arrow. In other words, the lead needle is preferably the lowermost needle of the slope set and in this way you can make a single line of color with the application being like placing “ducks in row”.

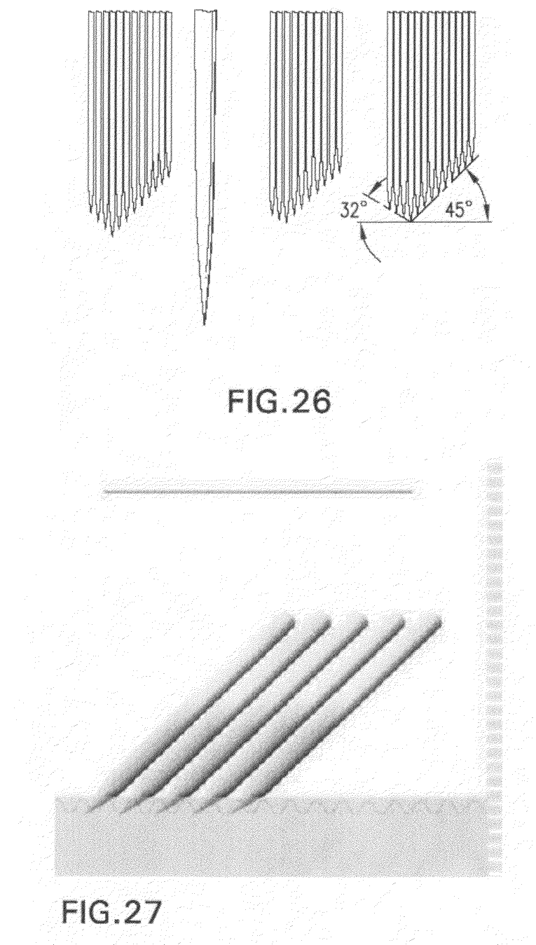

FIG. 5 illustrates a graduated lining (e.g., hairstroke) technique wherein, preferably without power, the skin is tapped with or without color. Preferably the needles are projecting the maximum distance out from the tip of the handpiece which is helpful when you want to be cautious as one can use this technique to tap in color in handtool fashion. Further note that, as with the FIG. 4 lining technique, the leading edge needle is the one opposite to the “toe” needle mentioned above relative to the sloped set of needles on the illustrated single slope edge presented for this embodiment. With reference again to FIG. 51 the lead needle is designated needle “1.” As can be seen from, for example, FIG. 26 at least some of the application techniques and possibly all (with a suitable vertex arrangement) can be achieved with a multi-slope needle such as those described in '115 application with FIG. 26 providing a few examples of needle devices with multi-slope edging falling within a single plane. This would provide, for example, to use with the same needle set a 3 point common level penetrating with proper orientation and by simple manipulation of the headpiece a 5 needle tip edge common level penetration under the various applications techniques featured under preferred embodiments of the present invention. Relative to the use of a multi-edge needle set there would be utilized an arrangement wherein the leading needle of the moving edge is the needle external to the longest extension intermediate needle in the vertex point or region. Note also from FIG. 5 the preference for some of the needles but preferably leaving at least two penetrating to be positioned above the skin surface which is helpful in a feathering action such as that shown in the uppermost line pattern shown in FIG. 5. Also, FIG. 21 shows needle sets suited for use under the present invention having three, four and five needle single edge slopes, respectively.

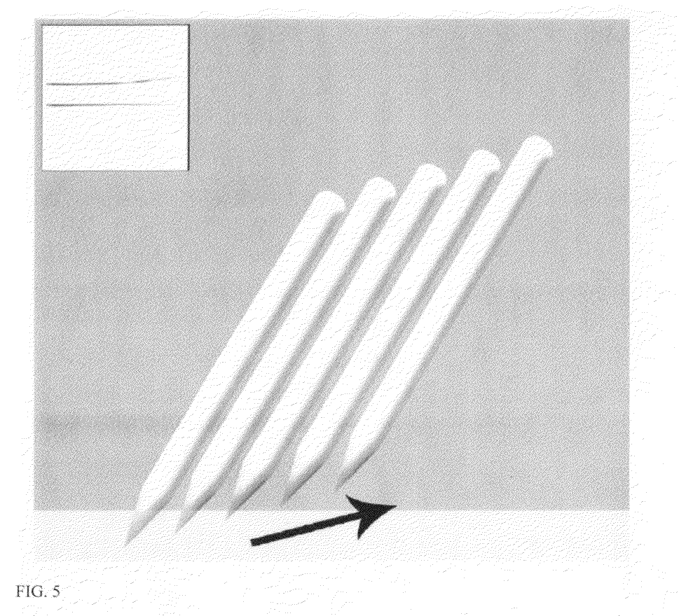

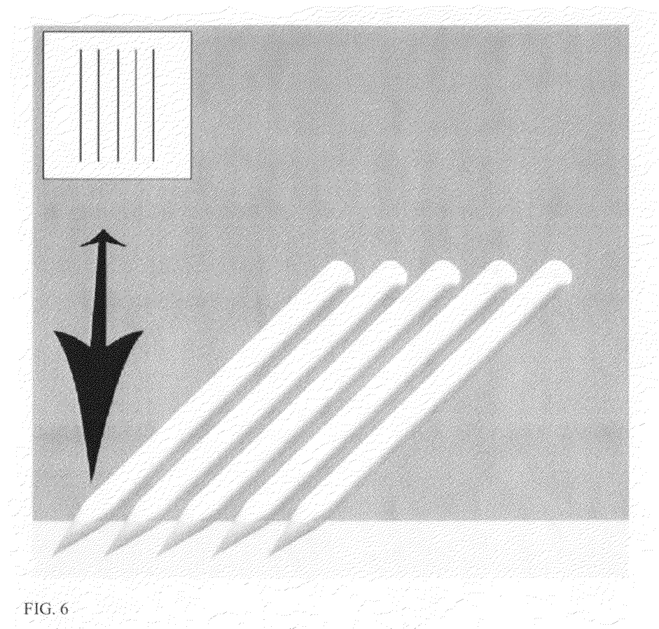

FIG. 6 illustrates a shading with the full width of the needles (as with the preferred technique shown with all needles extending to a common depth across width upon penetration). In this shading technique (preferably powered although non-powered is also an option as well as with any of the techniques mentioned herein). As seen from the footprint line pattern shown in FIG. 6, the needle set is moved back and forth (side-to-side) in a motion over the same area which provides for rapid and uniform shading.

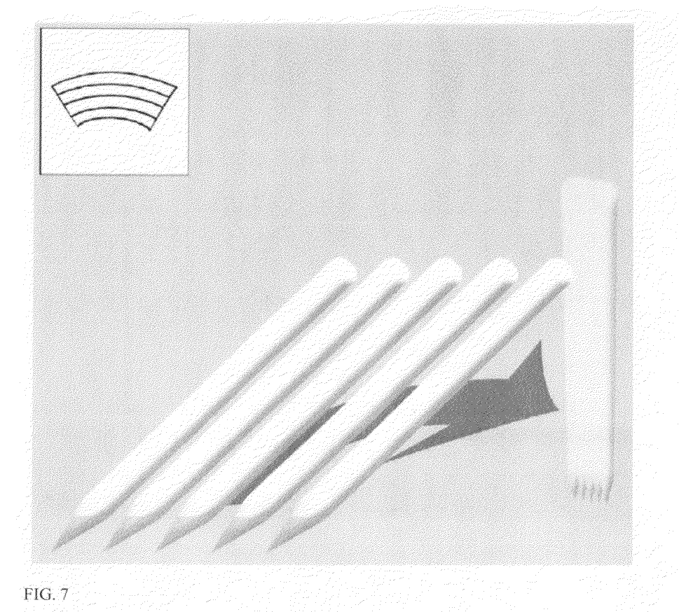

FIG. 7 shows a windshield wiper stroke or technique under an embodiment of the invention wherein, preferably without power (although a powered approach is an option), a user can tap skin with or without color as desired. In a preferred arrangement the needle set is arranged such that the needle “1” referenced in FIG. 51 is at the lower of the windshield arrangement and the toe needle or needle “5” is at the upper edge of the illustrated windshield pattern. Also the user preferably places the needle oriented as noted above in a center of the desired pattern application location with the wrist held relatively stationary and the hand rotated to achieve the noted pattern. With this orientation the user can easily insert to a common depth full needle set while also involving a windshield wiper type motion to cover the illustrated area. The sloped edge applied as shown in FIG. 7 is naturally suited for curves and thus is well suited for cupids brow and eyelids.

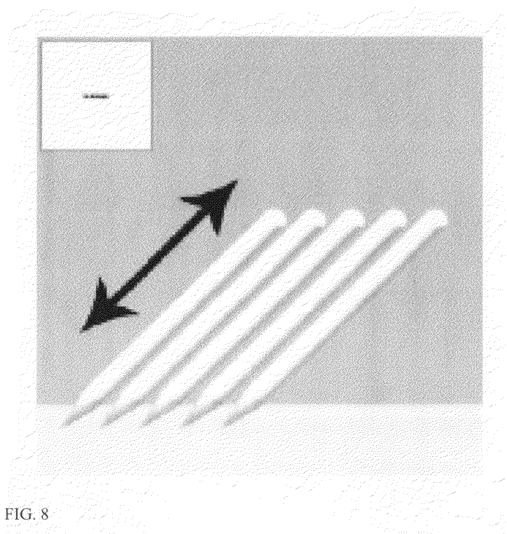

FIG. 8 shows a power tapping technique (preferably utilizing a powerized setting). As seen in the footprint depiction in FIG. 8 there is presented a fairly dark series of spaced apart points under the power tapping technique. For example, in an embodiment under this power tapping technique the skin is tapped into the skin about 5 to 8 times per area then the needle set is moved a little bit forward as would be done in a handtool operation. This technique is useful for getting color into the lower eyeliner and lip liner. Preferably the reciprocation machine is used with a 3 or 5 slope needle set with a large amount of color being applied (e.g., a needle moving into and out of the skin at 83 times per second over a desired period of time).

Furthermore, under the FIG. 8 technique there can be carried out “pointillism” which involves making dots with the needles in one spot. In other words, under this application is not moving the handpiece and the needles enter the skin multiple times in one spot. This is useful for marking the skin. For example, when you want to mark the upper eyelid outer “flare” the machine can be turned on while the noted needle configuration is applied as shown in the same spot as in, for example, a 45 degree upward and outward orientation relative to the outermost eyelash.

In addition, the FIG. 8 technique can also be carried out with the machine off and the needle set being used to dot or tap the skin, which is slower but can provide for greater control relative to ink insertion.

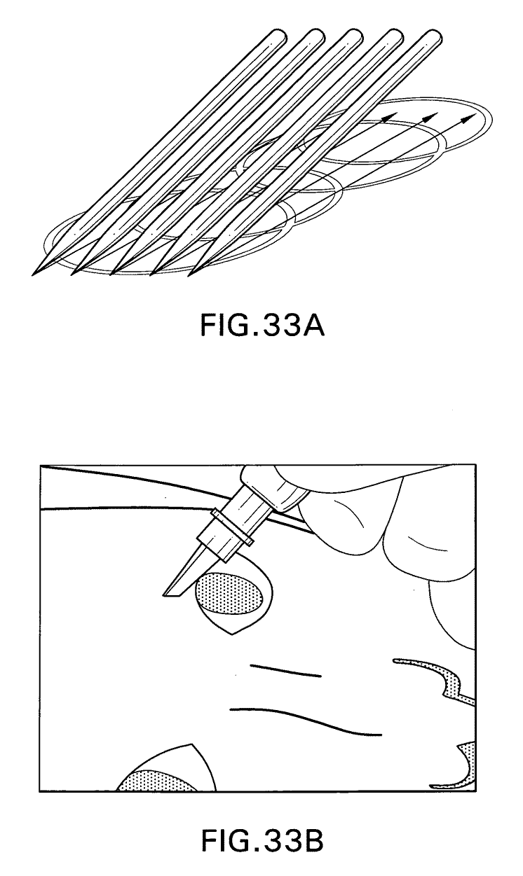

FIG. 9 shows a loop application using the same needle set as described above for the other figures wherein, with the power on, the operator makes circular loops in an oboviod fashion (that is under this technique it is preferable to move the needle set forward in the noted direction while conducting a circular movement in the handpiece. As further shown by the “stepped size” loop depictions suggesting movement into the paper, the edge is preferably moved transverse to the plane of the needle set when forming the loops (this is further illustrated in FIG. 33A).

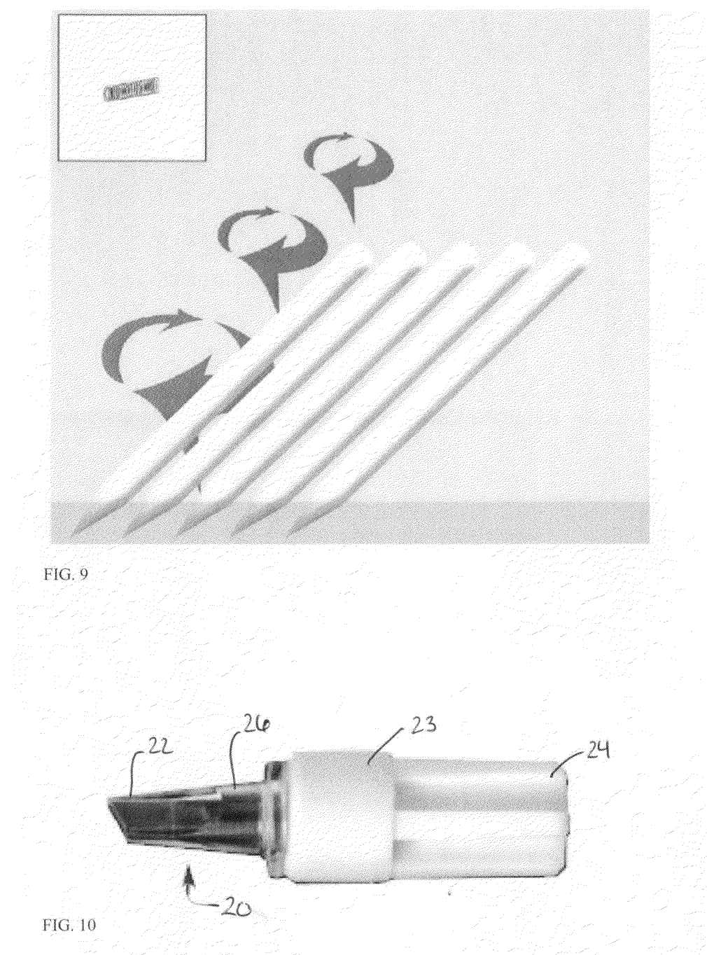

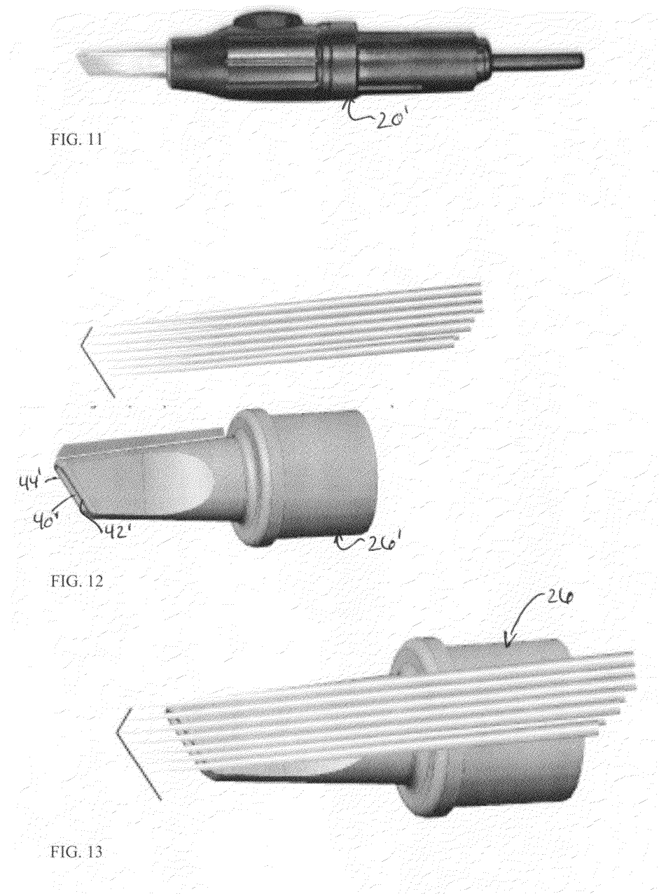

FIGS. 10 and 11 illustrate cartridges that are suited for use in a handpiece (e.g., a digital handpiece) of a mechanism to reciprocate the needle with power or in a non-motorized handpiece (e.g., pen-like support) which supports the needle and out from which the needle extends. As shown in FIG. 10, cartridge 20 has a distal, end 22 and a proximal end 24 having a suitable engagement configuration for attachment to a handpiece or like support. The distal end is structured depending upon the design of the receiving handpiece or alternate cartridge support structure. There is further provided intermediate area 23 which represents a transition area between ends 22 and 24 and which preferably supports the needle device's base in relatively fixed fashion and which is preferably designed for providing a stop to the longitudinal insertion of the cartridge into a receiving means of a handpiece or similar support means.

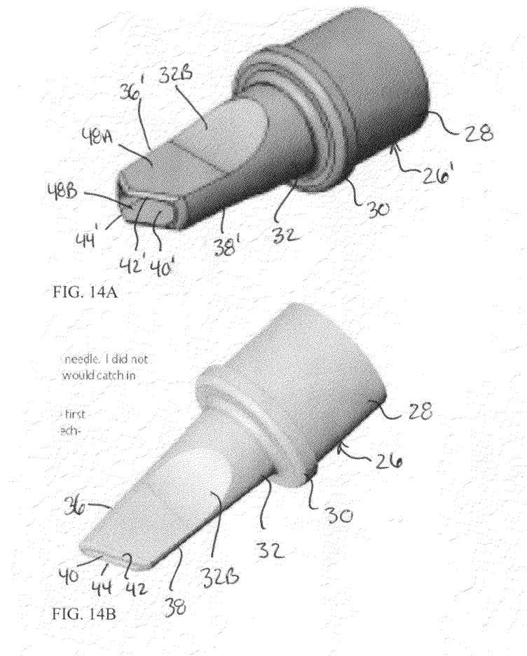

With reference to FIGS. 10, 13 and 14B there is illustrated a single slope edge needle set holder tip 26 designed to receive all of the length of the needles in the set but for an outmost end region of those needles with outermost end region extends to the tips of the needles in the set. As best seen by FIG. 14B holder tip 26 preferably has a proximal base section 28 designed for a preferred releasable, securement reception in a conforming portion of the distal end of a cartridge such as that shown in FIGS. 10 and 11. In addition, as further shown in FIG. 14B there is preferably a collar section 30 providing an abutment location for the inserted holder tip relative to the cartridge. Further the needle conformance region of the holder tip preferably includes a sloping transition section 32 which has a cylindrical base section 32A followed by a pinch or inward sloping section wall set (only one shown) 32B. The inward sloping section extends into a parallel set of side walls (one shown) 34 having connecting, exterior curved wall edges 36 and 38. Also, the distal end of the curved edges and the side walls 34 there is an opening 40 through which the tips of the needles extend. Further the free edges 42 and 44 of the wall set 34 preferably have at that opening a slope corresponding to the slope of the needle set's sloped edge. In other words, FIG. 10 shows a needle cartridge with a clear holder tip having a sloped free edge (opening defining rim) out from which the needles of the needle device preferably extend a common distance. Thus, the free edge rims at the distal end of the holder tip extend parallel to the needle tip edge. The holder tip also preferably has a relatively universal connector end (e.g., handheld and/or powered tool connection). Also, FIG. 25 shows a single line edge set of needles having a preferred slope angle and shown housed in a holder tip and extending into the skin such that the needle tips reach a common depth and the holder tip free edge is above the skin's exterior surface (shown planar here).

FIG. 11 shows an alternate cartridge design 20′ suited for attachment to a handpiece having a conforming capture means for capturing the cartridge and providing for appropriate reciprocation of the needle tips. For additional discussion of motorized needle reciprocation mechanisms reference is made to the previously noted copending application Ser. No. 10/677,115.

Further, FIG. 12 shows a modification schematic for altering a holder tip for a single sloped edge needle set to a multi-edge sloped needle set such as the double and triple edge embodiments described in the noted copending application which modification provides a holder tip to provide for its peripheral opening 40′ defining edges 42′ and 44′ presenting two parallel opening defining edges so as to have two parallel rim edges coinciding with the needle slope edges. In other words, FIG. 11 shows a needle cartridge with a holder tip having a sloped free edge out from which the needles of the needle device extend a common distance and parallel to the needle tip line plane formed by the tips of the needles in the needle device as well as a hybrid connector end for connection with, for example, a handheld and/or powered tool as in a digital machine for powered needle reciprocation or skin penetration movement. In other words, FIG. 12 shows an example of a holder tip providing for a two line tip plane or two-edge needle device shown above the holder tip. FIG. 13 shows the needle set of FIG. 12 superimposed upon a single sloped holder tip to illustrate, while less preferable, a holder tip's distal end need not always have mutually parallel rim edgings defining the needle extension opening.

Cartridges such as that shown in FIGS. 10 and 11 are suited for preexisting reciprocating machines and associated handpieces as MTDerm, Harmonix, Permapoint, BocaTa2, Blue and other handpieces and rotary machines as in KP, Infinite and Freedom.

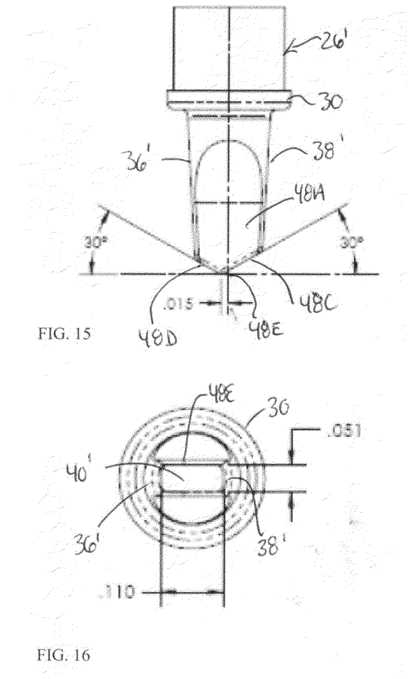

FIG. 14A shows a perspective view of holder tip 26′ represented by the schematic reconfiguration depiction shown in FIG. 12 with its peripheral edges or rims 42′ and 44′ at opening 40′ and with a preferred embodiment featuring opposing walls 48A and 48B with each defining a pair of sloping rim sections as represented by 48C and 48D in FIG. 15 which extend out from a vertex rim section 48E. FIG. 14A further illustrates the curving edge walls 36′ and 38′ completing the surrounding enclosure relation to the received needle set. Further the rim sections 48C and 48D are preferably design to generally conform in slope angle with the respective needle sets sloping edges as represented by FIG. 12 with the noted conforming edging. For example, the needle set of FIG. 12 is shown superimposed (in use would be received internally to the holder tip) relative to the modified tip holder schematically presented in FIG. 12; with the free sloped edging of the holder tip being in common with the edge slopes of the two edge needle device shown (an even greater number edge needle device is preferably received in an equal number free, sloped edge holder tip with the tip ends of each needle preferably extending out a common amount within each subgroup relative to the corresponding free edge slope of the holder tip). Also as shown in FIG. 15 there is provided a holder tip vertex accommodation point or edge region (in this case there is featured a vertex edge region which is of a length designed to conform with the vertex edge region as in the far right in FIG. 26 with a non-single vertex point arrangement). FIG. 15 further illustrates an illustrative (non-limiting) holder tip vertex edge accommodation length of 0.015 inches. This arrangement shown in FIG. 15 can also be utilized for the intermediate illustrated FIG. 26 embodiment with single point vertex needle arrangement or a conforming sharper angle extremity in the holder tip can be utilized at the vertex point.

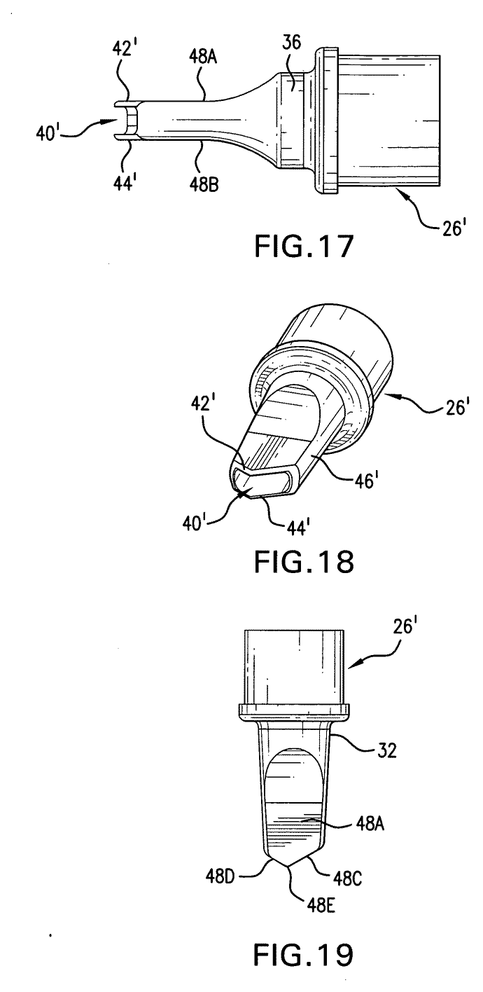

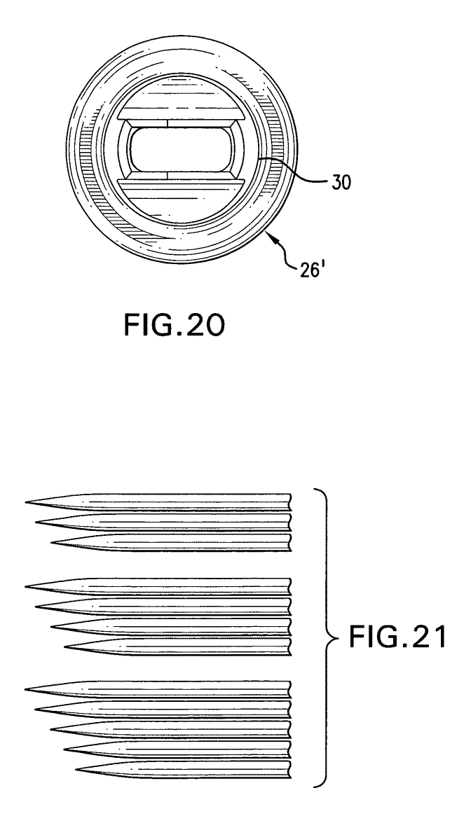

FIGS. 15 to 19 show some additional views of the holder tip 26′ (shown non-transparent but is preferably formed of a transparent plastic material for improved needle set visualization). FIGS. 15 and 16 further show some angle and extension length parameters which are not meant to be limiting but illustrative of some suitable dimensions for an embodiment of the present invention. This includes some rim edge opening sloped (e.g., 30 degree sloped rim walls for edges 48A and 48B although they can also be unequal in slope as when conforming to unequal slope angles of a received multi-edge needle set as well as a 0.110 inch width at the distal end of edges 36′ and 38′).

The needle device of the present invention is preferably received within a holder tip such as those depicted in FIGS. 12 to 14B with the free edge(s) of the holder tip preferably being equal to the line or edge formed by the tip ends (all tips within a group or subgroup defining edge preferably extend a common distance from the corresponding free edge). Furthermore, in the discussion below, a 32° slope from the horizontal is described as being a preferred angle well suited for use in the application methods of the present invention. While a variety of different angle slopes (e.g., ±7° from the noted 32°, there is preferably utilized a range of 32°±5° and more preferably 32°±2°).

The holder tip design is thus suited for a multi needle insert as represented above as in where 5 needles are provided which present a single sloped edge. An example of this can be seen in FIGS. 51 and 52 showing a transparent holder tip generally similar to that described above but having only one angle of sloping side rim edges defining the needle tip access openings.

FIG. 38 illustrates an illustration of a powerized needle reciprocation kit 60 with main housing 61 for the motor and controls (not shown) as well as an electrically connected handpiece 62 having reception means for receiving a cartridge like that of FIG. 11 (a sealed three pack of the same represented by 63 in FIG. 38). The noted handpiece 62 which receives the needle device cartridges is well suited for use in the method of the present invention in both power on and power off states depending on the application step involved. FIG. 38 also illustrates an electric power adaptor 64 and a foot pedal 66 for adjusting the reciprocation speed of the supported needle set when in use. The above described cartridges and holder tips have been designed for use in machines such as the Harmonix Machine of German Digital. Under preferred “powered” embodiments using devices such as those described above which provide power based reciprocating needle implantation, the needle(s) enter and exit the skin at a rate of approximately 60-200 strokes/second.

It is further noted that under the present invention the needles sets featured herein can also be utilized in pen machines as in those that take a 2 inch needle set.

FIG. 27 shows an example of a uniform line application technique featuring all needles touching the skin at the same depth and same time with a five needle set being illustrated. As seen in FIG. 27 there is formed a uniform and continuous line through the technique of providing for example a 32 degree slope in the needle and penetrating the skin to a desired depth while maintaining the needle edge parallel to the penetrated skin surface which is facilitated by way of the design of the needle set and cartridge holder (or alternate support means) which preferably present a holder tip with a conforming rim edge slope to facilitate making such a parallel and even depth insertion into the skin.

FIG. 28 shows a schematic example of a graduated line formation technique (either straight line or flared at end) well suited for eyebrow formation and using the same five needle set as in FIG. 27. As shown in FIG. 28 the line is preferably formed under this technique to go from thick to thin as in a calligraphy technique with the thus formed “hairs” being formed thickest at the base and made to thin out at the end. Thus, this technique is well suited for forming eyebrow hair strokes (as opposed to “picket fences” formed by other techniques). There is preferably an application of 3 times per hair while avoiding cutting the skin with the needle.

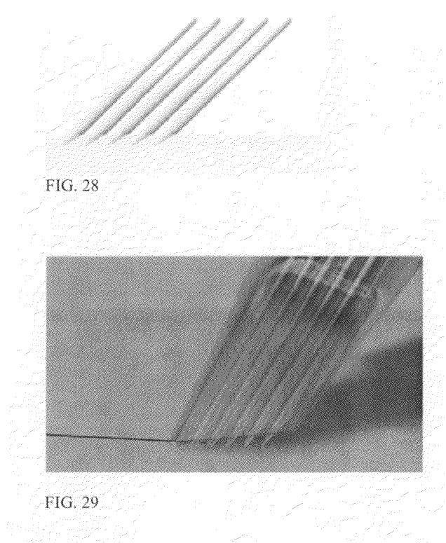

FIG. 29 shows an example of the graduated line technique shown in FIG. 28 on a skin surface presentation and the line being formed as the needle set is moved (reciprocates and moved by the operator by grasping the handpiece).

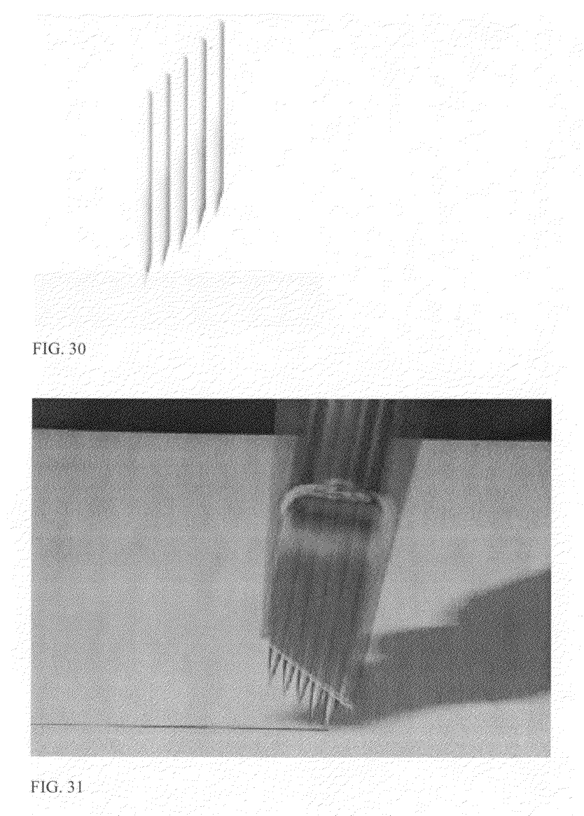

FIG. 30 shows a schematic view of the same needle set utilized in FIGS. 28 in the above described “ballerina” technique featuring a single needle skin penetration amongst the five needle set. The related FIG. 31 shows an example of the ballerina technique of FIG. 30 on a skin surface presentation being reciprocated and with the clear holder tip visible. A rotation technique would be carried out from the position shown in FIG. 31.

Thus FIGS. 27 to 31 illustrate techniques suited for use in lining (e.g., one of the many different techniques afforded by the needle arrangement of preferred embodiments as in 9 to 15 different techniques). That is, FIGS. 27 to 31 illustrate lining with a uniform line (using all needles in a row to make a line of equal thickness); graduated line (thicker at the beginning and thinning out at the end with an added flare or no flare) and single needle for fine lines wherein the top needle is used like a ballerina on toe with fine “tweaking” made possible.

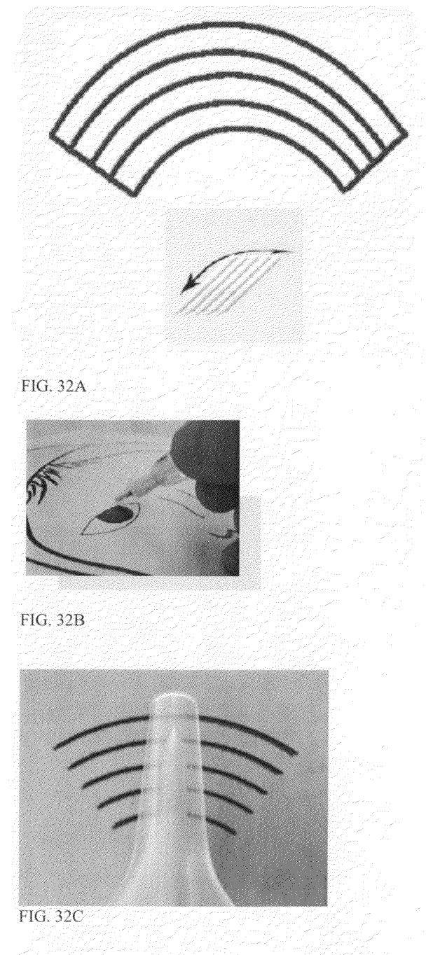

FIG. 32A shows a schematic presentation of the windshield wiper stroke or technique (a shading technique) showing the needle set and pattern produced as when the wrist is held stationary at the center of the stoke and the hand is rotated to produce the illustrated pattern. FIG. 32B shows the windshield wiper technique in an eyeliner training setting. FIG. 32C shows FIG. 32C shows an example of the windshield wiper technique on a skin surface presentation and with the clear holder tip visible. As the slope of the needle set is naturally suited for curves, this technique is well suited for use in applications such as cupids bow and eyelids (e.g., eyeliner).

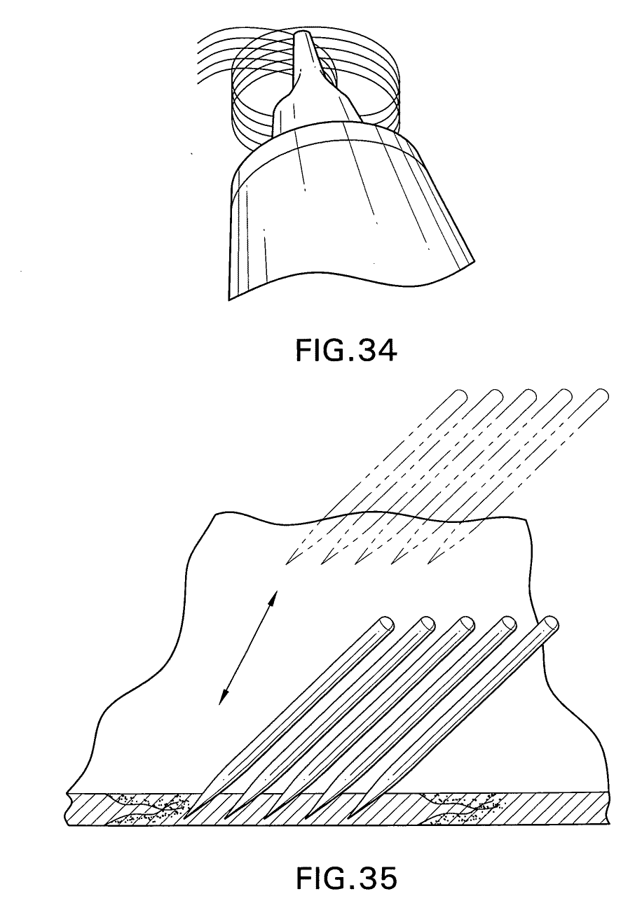

FIG. 33A shows a schematic presentation of the looping or obovoids formation motion (loose circle) stroke or technique showing the needle set and a schematic of the pattern produced (an additional shading technique). As seen by FIG. 33A the full width is adjusted across in combination with a looping motion in the needle set (note the directional arrows with overlay of loop rotation). FIG. 33B shows the loop technique in an eyeliner training or practice setting. FIG. 34 shows the loop technique in process relative to a skin penetration surface. There is preferably avoided tight circle looping as that can cause trauma as it introduces a drill type action on the skin. An operator can tell if there has been overworking if serum (clear yellow fluid) is visible. If so, the application can be stopped and check made as to the needle depth and possibly other settings.

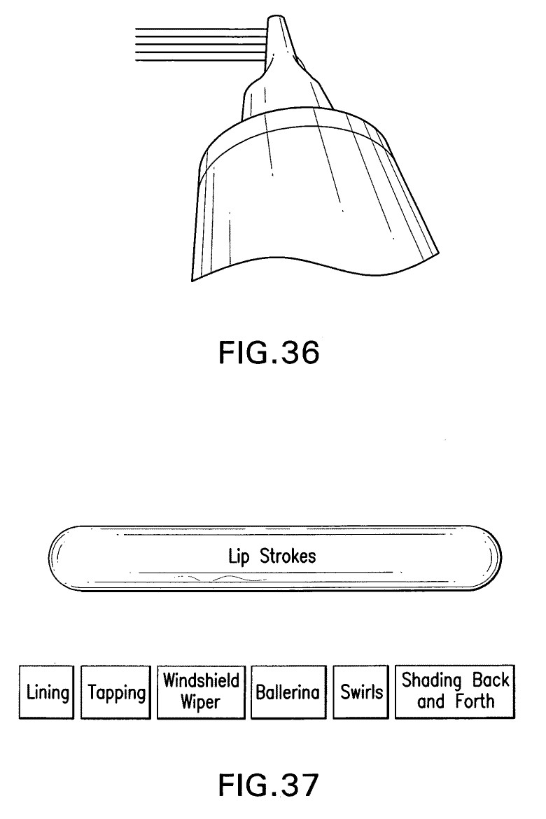

FIG. 35 shows a side to side technique with FIG. 36 showing the side to side shading technique of FIG. 35 in process. Under this technique there is featured a back and forth movement to put in color with all needles preferably touching skin (e.g. a 32 degree angle needle set) in essentially brush like fashion). The side to side shading technique is suited for use in filling in areas of the lip and areola. Further such a technique can be utilized for brow fill in (back to back or side to side motion). Further for a softer color under this technique there can be applied one or two drops of saline or eyewash to the brow pigments (mixed thoroughly) and then there is shaded over hairstrokes earlier applied.

FIGS. 32A to 36 are illustrative of shading techniques useful for filling in areas with color as in lipcolor, areolas, thick eyeliner, smudgy eyeliner and some brow color.

The shading strokes are thus inclusive of a windshield wiper technique, a back and forth or side to side technique and an obovoids or looped circle technique.

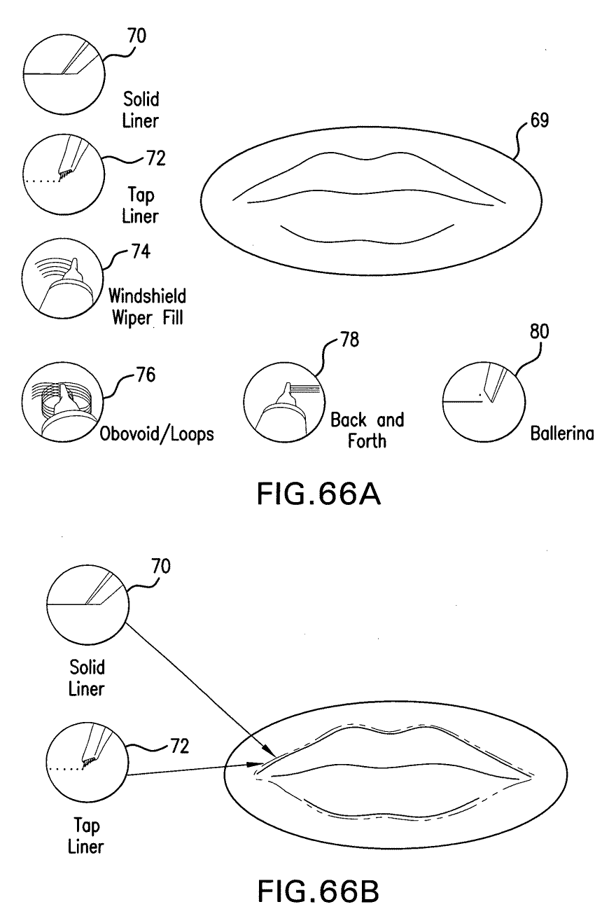

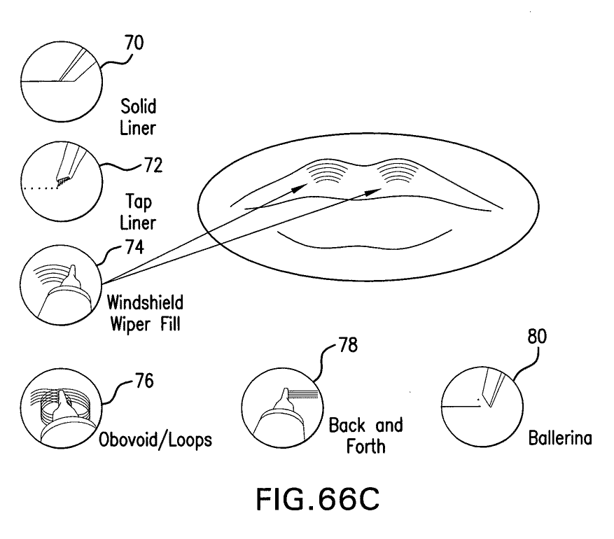

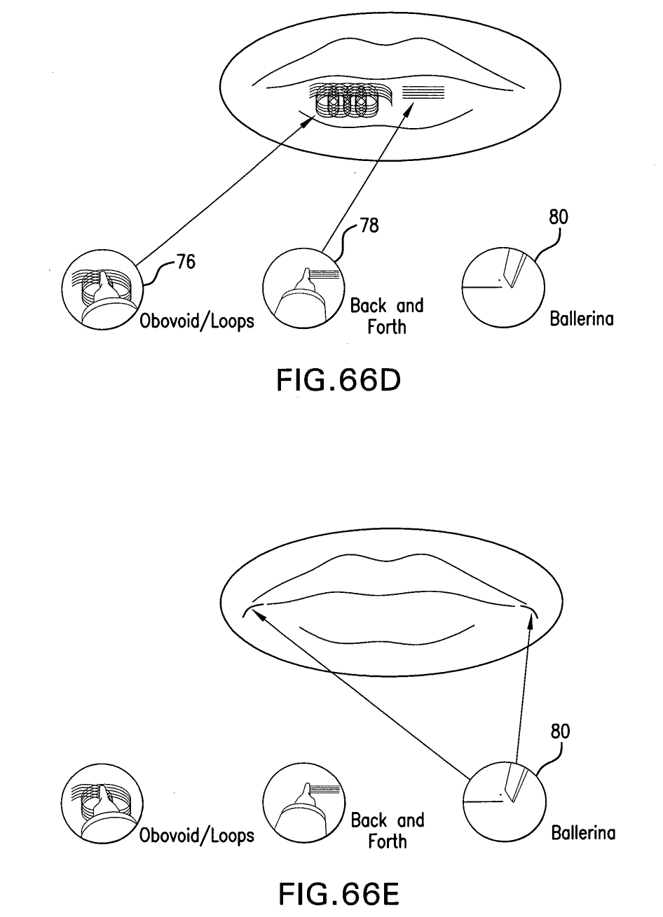

FIG. 37 shows a preferred stroke schedule for lip application with FIGS. 66A to 66E providing additional detail. FIG. 37 shows in general some of the strokes or techniques well suited for lip applications. FIG. 66A provides a preferred stroke lip application process illustration to the lip(s) 69 of a subject which features the following strokes or techniques (preferably in the noted sequence although alternate sequencing is also featured under the present application) solid liner 70, tap liner 72, windshield wiper 74, obovoid or loops 76, back and forth 78 and ballerina 80. Additional single point marking may also be desirable to provide additional end or starting point guidance.

FIG. 66B shows lip lining tapping and lining featuring solid liner and tap liner to provide the noted lip outline, which is preferably techniques carried out initially to set the framework or foundation for the later applications

FIG. 66C shows the use of the windshield wiper technique including application to the two larger regions of the upper lip to opposite sides of the upper lips central constricted region as shown (Cupids Bow region). This is preferably carried out as an initial intermediary step relative to the steps featured in FIG. 66B and those described below in FIGS. 66D and 66E.

FIG. 66D shows shading the lower lip with a combination of back and forth and looping techniques. This is preferably carried out as a secondary intermediary step relative to the steps featured in FIGS. 66B and 66C and those described below in FIG. 66E. although alternate sequencing is also featured under the present invention such as switching in order steps shown in FIG. 66C and 66D as but one example.

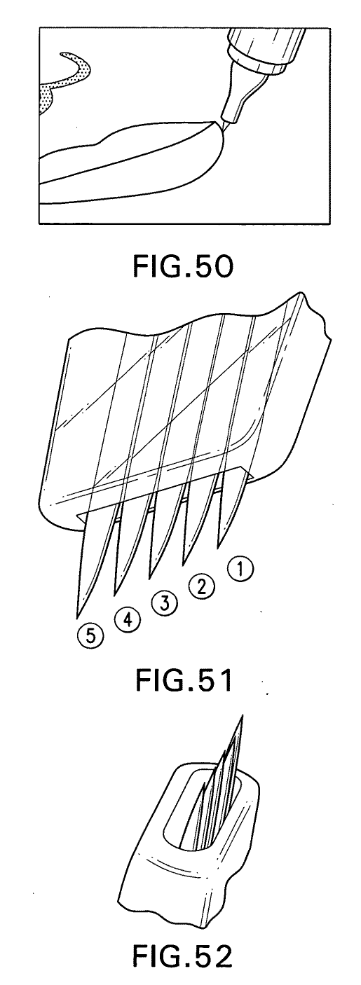

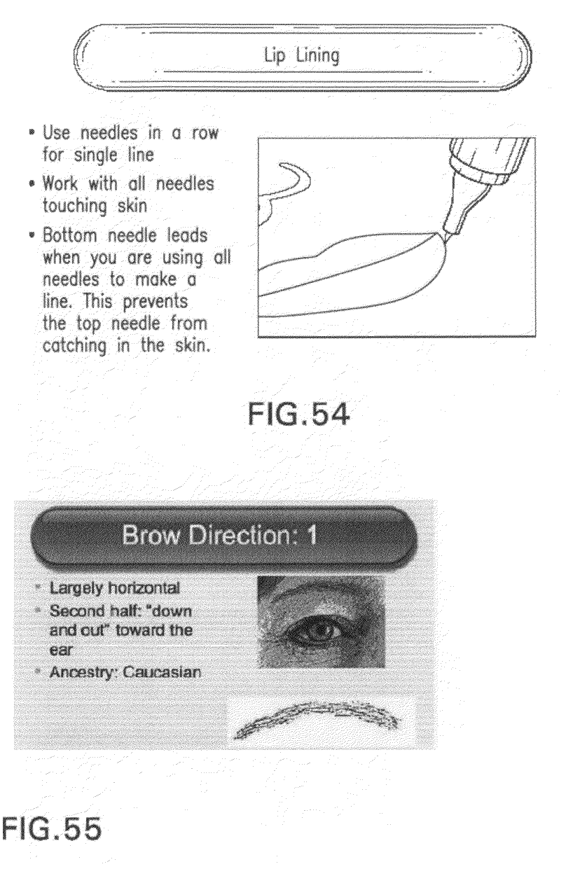

FIG. 66E shows corner and fine finishing applications using the ballerina technique. Reference is also made to FIGS. 50 and 54 shows a lip lining training or practice examples.

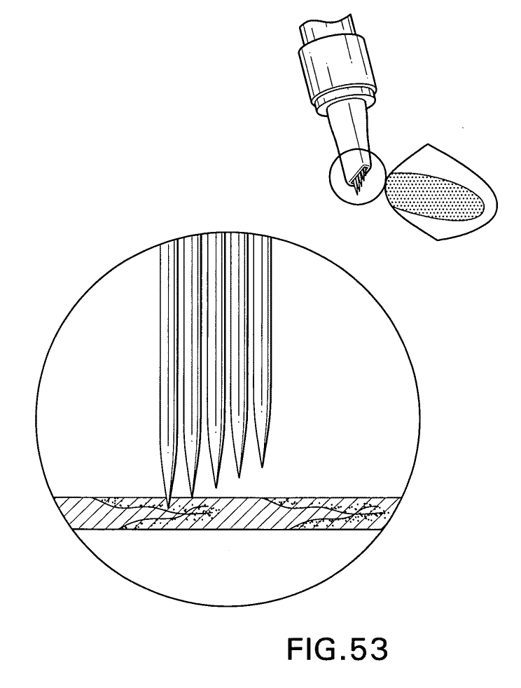

With reference to FIG. 53 and FIG. 65 there is provided a discussion of an example of suitable eyeliner strokes using strokes or techniques described herein. FIG. 53 shows the ballerina technique training or practice example wherein there is used the highest needle alone such that only one needle is brought in contact with the skin during this technique. FIG. 65 shows a preferred stroke series for eyelid liner applications (preferably in the noted sequence although alternate sequencing is also featured under the present application). As shown in FIG. 65 there is featured an eyeliner application stroke approach for eyeliner 80 which includes the illustrated strokes of solid liner 82, tap liner 84, ballerina 86, windshield wiper 88 and back and forth (side to side) 90. Reference is also made to FIG. 61 showing an angle approach relative to the upper eyelid with the application being made for smudgy eyeliner, medium thick or very thick eyeliner with rotational use of needles with hand.

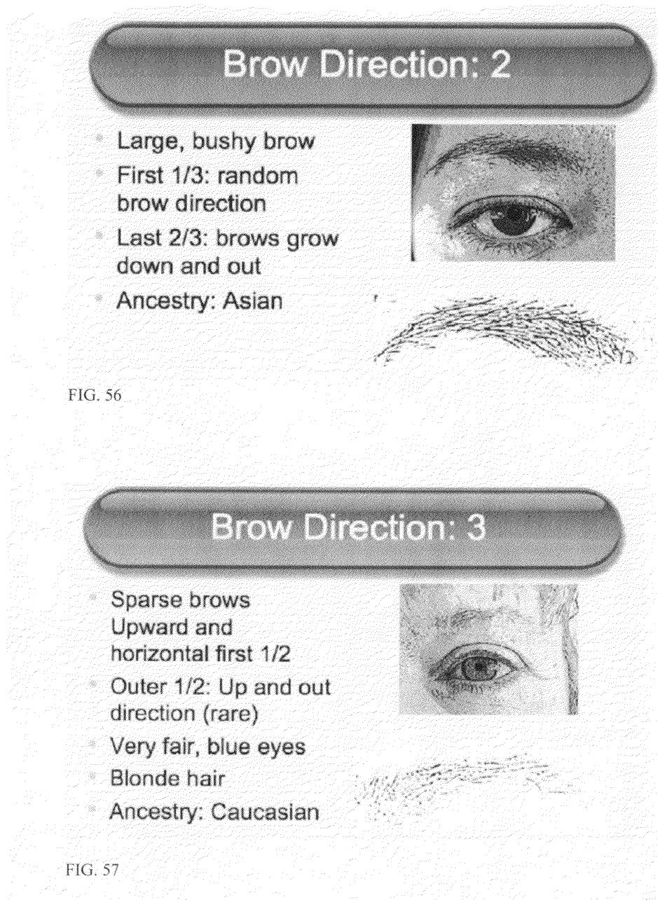

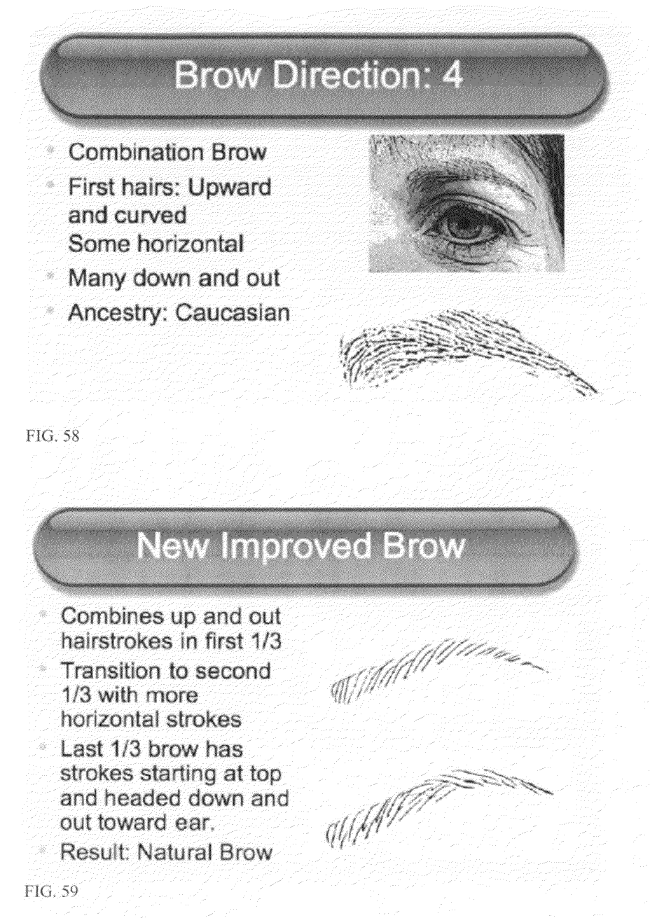

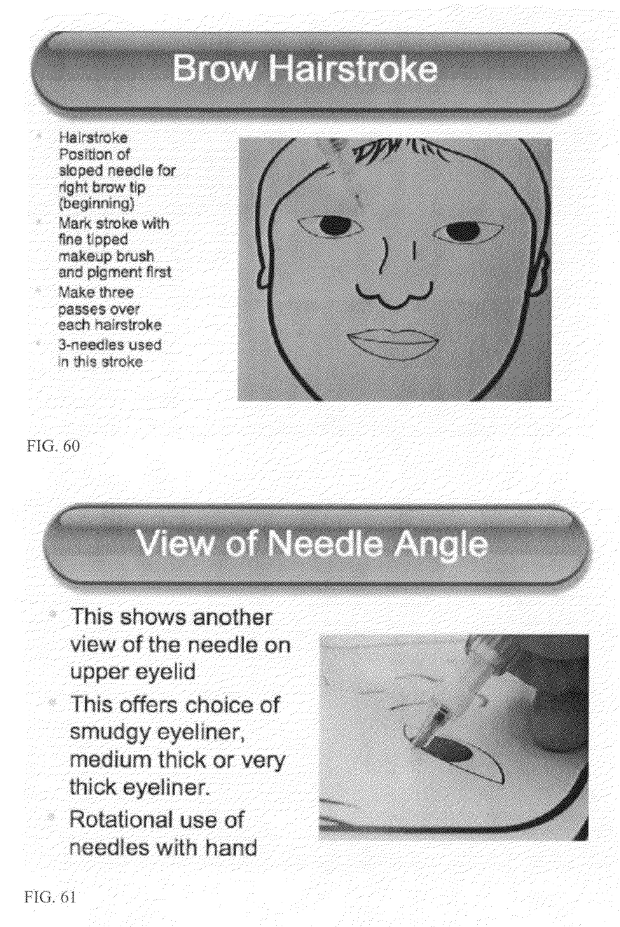

FIG. 55 to 58 show first to fourth brow direction application example for subjects of different ancestry, respectively. FIG. 59 shows an alternate brow application technique, while FIG. 60 shows a hairstroke application to the brow.

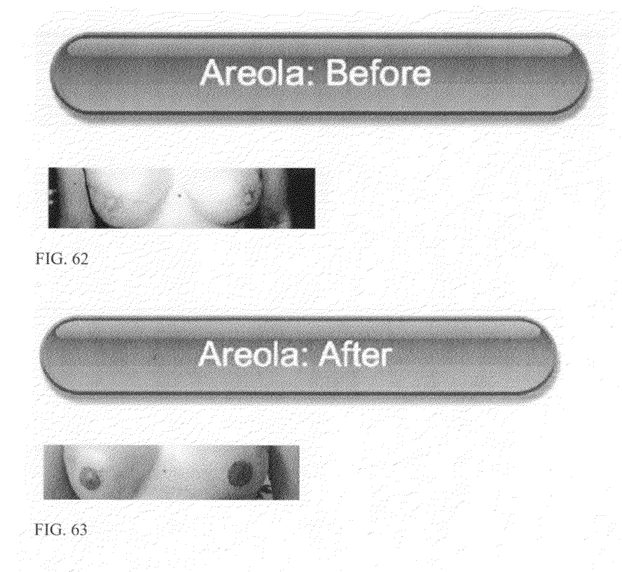

FIGS. 62 and 63 illustrate before and after views following a needle application technique using an embodiment of the present invention with gentle sloped needle sets. That is, ordinary needles and handtools place the needles at various depths over a curved surface such as a breast. A sloped needle set such as referenced herein under the present invention are well suited for dealing with scars on the believed basis that the “comb” the tissue at a more superficial depth. The areola is three dimensional with its round shape being readily handled under the needle configurations of the present application. For example, it is considered that the sloped needle fans over areas putting in evenly and quickly color and shading is easily achieved using one or more of the shading techniques described herein.

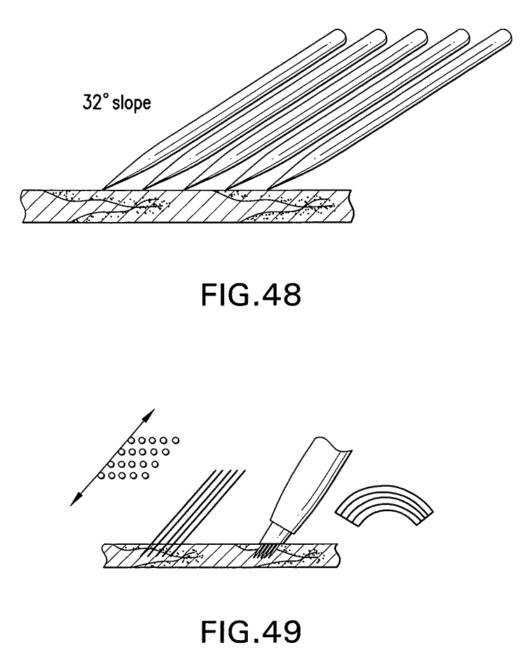

The gentle sloped needle arrangement of the present invention (preferably a single slope edge of about 32 degrees as shown in FIG. 48 and for which slope desirable footprint patterns can be achieved as shown in FIG. 49) is also well suited for application on scars, correction and camouflaging applications

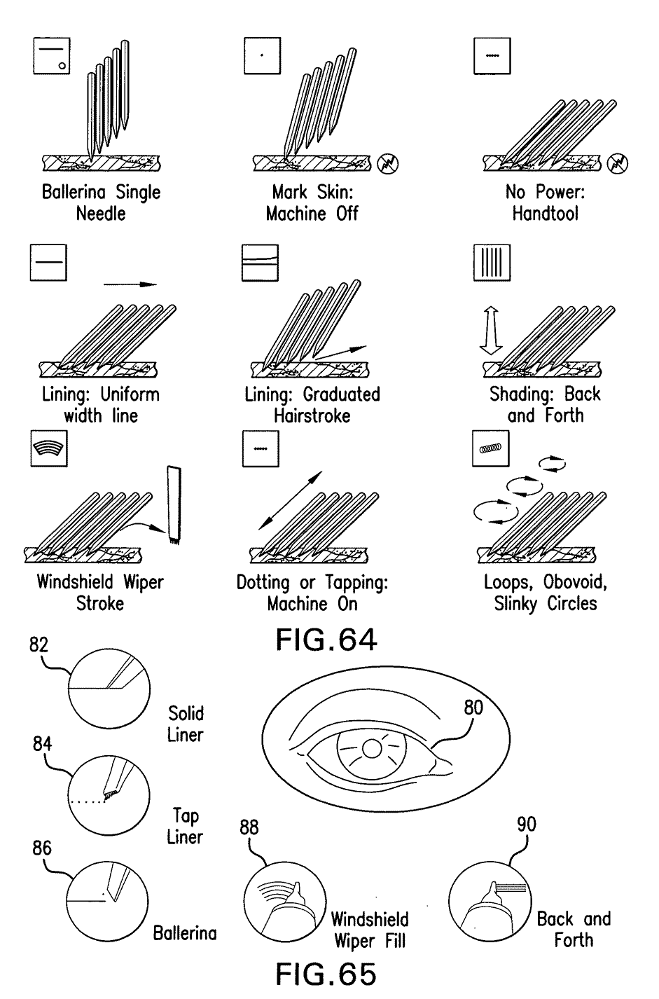

FIG. 64 provides a consolidated view of some of the preferred application techniques of the present invention with
(i) the illustrated “ballerina (with adjustment)”, uniform lining and graduated hairstroke lining being examples of “lining techniques”;
(ii) with the windshield wiper stroke, back and forth shading and obovoid application techniques being illustrative of “shading or filling with color techniques”,
(iii) with the tapping (with machine on) being illustrative of a “power tapping” application technique, and
(iv) with the handtool or no power tapping application technique (e.g., similar to power tapping without power utilized) and the “mark skin” with machine off being illustrative of “power off” application techniques.

Provided below in Table I is an example of some application combinations featured under application techniques embodiments for achieving a desired application process which are not meant to be limiting but illustrative of some efficient combinations for achieving the indicated application processes (e.g., in some settings some of the application techniques not indicated for use may prove useful while others indicated as being utilized in an application process may be dropped depending on the circumstances (e.g., recipients skin characteristics or anatomical arrangement, etc.). For example, a non-powered mark technique can be useful to help in guidance particularly for those less practiced in application techniques.

TABLE I

| Technique Description | | | |
|---|---|---|---|
| (preferably with a 3 | | | |
| to 5 single slope | | | |
| needle set or hybrid | Lip | Eye Application | Brow |
| multi-edge needle set) | Application | (e.g., eyeliner) | Application |
| Tapping (machine or | x | x (e.g., tap liner) | x |
| non-machine) | | | |
| Lining (uniform width | x | x (e.g., solid liner) | |
| line) | | | |
| Lining (graduated as | | | x |
| in graduated | | | |
| hairstroke) | | | |
| Ballerina | x | x | x |
| Side to Side (Back | x | x | x |
| and Forth) | | | |
| Obovoids | x | | |
| Windshield Wiper | x | x | x |
| Marking (non- | x | x | x |
| powered) (used, for | | | |
| example, if guidance | | | |
| marking deemed | | | |
| desirable as in lip | | | |
| treatment lining | | | |
| guidance marking) | | | |

It should be emphasized that the above-described embodiments of the present invention, particularly, any “preferred” embodiments, are merely possible examples of implementations, merely set forth for a clear understanding of the principles of the invention. Many variations and modifications may be made to the above-described embodiment(s) of the invention without departing substantially from the spirit and principles of the invention. All such modifications and variations are intended to be included herein within the scope of this disclosure and the present invention.

What is claimed is:

1. A method for introducing a needle device into a subject comprising:
causing a single needle device comprised of a plurality of needles arranged at a gentle slant, preferably 30-35°, to enter and exit a layer of the subject, , wherein the needles enter the layer at the same depth at the same time at, for example, a 32° angle, and
moving the device across the layer to form a needle implantation pattern.

2. The method of claim 1 wherein the subject is a human and the layer is a skin layer and the needles enter and exit the skin at a rate of approximately 60-200 strokes/second.

3. The method of claim 1 wherein the subject is a human and the layer is a skin layer and the method further comprising supporting a color agent with the needle device for implantation within the skin and using the same needle device to perform a lining and a shading application technique to achieve different implantation patterns using both a powered reciprocation needle implantation mode and a non-power needle implantation mode.

4. The method of claim 1 wherein the needle device supports a desired substance which is selected from ink, dye, pigment or other intradermal marking means and the layer is a skin layer.

5. The method of claim 4 wherein the needle point level category is chosen as the superficial papillary dermis.

6. The method of claim 1 wherein the site of delivery includes lips, breasts, camouflage, eyebrows, medium to thick eyeliner, and smudgy eyeliner and eyeshadow.

7. The method of claim 1 wherein the layer is a skin layer and movement of the needle device is a method technique where a single point of the multi-needle device is inserted within the layer and the remaining non-inserted tips are rotated in “ballerina” fashion forming a footprint in the skin.

8. The method of claim 7 wherein the single point is represented by an outermost tip, relative to a support structure holding the needle device, amongst a sloped line edge of needles in said needle device.

9. The method of claim 8 wherein said ballerina rotation is repeated along a series of points arranged in a line in the skin of the subject.

10. The method of claim 1 wherein the layer is the skin of a human and movement of the device is a technique with multiple-points of the needles of said needle device being inserted within the skin at an angle relative to the surface of the skin with the slant edge defined by the needle tips of the needle device arranged parallel to the skin surface below so as to place the tips at a common level below the outer exposed surface of the skin.

11. The method of claim 10 wherein the technique is a “lining” technique resulting from the repeated insertion of one or more of needles to form the foot print.

12. The method of claim 10 wherein there is implemented an addition pattern movement in the needle device involving a technique wherein there is formed a graduating line in the skin by insertion of only some of the needle tips, and with some of the tips going to a different level of insertion.

13. The method of claim 12 wherein there is a flaring at the end of the graduated line by adjustment of some of the previously non-implanted needles into a penetration setting at an end of the graduated line.

14. The method of claim 10 wherein the movement of the device is a technique with formation of a plurality of lines in parallel fashion based on back and forth shading movement of the needle device.

15. The method of claim 10 wherein wherein the movement of the device involves a dotting or tapping function with reciprocated movement along the axis of the needles in the needle device.

16. The method of claim 1 wherein the movement of the device involves a windshield wiper stroke resulting from a rotation with displacement type effect placed on the set of needle tips insertions from a central region to opposite sides.

17. The method of claim 1 wherein the movement of the device involves a loop or obovoid sequence of movement in the needle device having all tips inserted to essentially equal depth during the course of the application process.

18. The method of claim 1 wherein the layer is a skin area of the lips and wherein the movement of the device includes multiple stroke techniques each using the same needle device and which stroke techniques comprise the following application techniques:
Liner;
Windshield wiper to fill cupid's bow on upper lip at midline;
Ballerina single needle used to do fine lining and finishing of corners of lips and cupid's bow;
Side-to-Side for shading and filling in color;
Obovoids or circles for filling in color;
Tapping with a needle reciprocation machine on for wide lip line; and
where the above application techniques can be performed in the order specified or as desired.

19. The method of claim 1 wherein the layer is a skin area extending about the eyes and wherein the movement of the device includes multiple stroke techniques each using the same needle device and which stroke techniques include each of the following:
Tapping for upper or lower eyeliner;
Side-to-side for upper eyelid outer ⅓ to “flare” and make eyes appear larger;
Windshield wiper for upper eyeliner “smudgy” effect;
Liner for narrower line that tapers toward nose;
Single needle, Ballerina, for very fine dotting or lining, especially toward inside eye toward nose; and
where the application techniques can be performed in the order specified or as desired.

20. The method of claim 1 wherein the layer is a skin area of the brows and the movement of the device includes multiple stroke techniques each using the same needle device and which stroke techniques include each of the following:
Graduated hairstroke: Line is thicker and become thinner like a real hair as in a calligraphy;
Sweep needles up full width makes 5 hairstrokes at one time;
Shading with Back-and-forth, or Side-to-Side Stroke;
Line where the needles are rotated 45° from wide to narrow toward the end of the eyebrow known as the “tail” and at full rotation the needles form a single line like ducks in a row; and
where the application techniques can be preformed in the order specified or as desired.

21. The method of claim 1 wherein the layer is a skin layer and said needle device is supported in a holder device having a distal end opening defined by holder tip edging which edging slopes in a common fashion as the slope of an adjacent needle tip defined sloped line edge and the holder device is maintained above or in contact with the skin layer during needle implantation.

22. The method of claim 21 wherein the needle device has a plurality of needle tip defined sloped edges lying in a common plane and wherein the distal end of the holder tip has a plurality of conforming slope edge regions relative to respective ones of the plurality of needle tip defined sloped edges.

23. The method of claim 1 wherein the needle device has 5 or more needles in a common plane needle set with those 5 or more needles defining at least one single edge tip line.

24. The method of claim 1 wherein the layer is a skin layer and the needle device is received in a holder tip which accommodates a two edge needle device having a common vertex tip or region and two needle tip defined sloped edges, with the lines or those edges extending thereout from a center needle, and wherein the holder tip has correspondingly sloped edges defining an opening at a distal end of the holder tip which is placed adjacent the skin during pattern formation.

25. A method for providing an intradermal coloring application process, comprising:
using a single needle device having three or more needles which extend out from a needle device support and wherein said needles are arranged along a common plane and have tips which define a sloped edge of about a 30 to 35 degree angle;
arranging said needle device as to have said sloped edge parallel to an intended skin penetration area;
implanting said needle device into the intended skin penetration area as to have the needle tips of the needles extend to a common depth and to place a common layer of skin coloration material at that depth;
manipulating said needle device using a variety of application techniques to form a variety of footprint patterns in the intended skin penetration area.

26. The method of claim 25 wherein in the application techniques includes at least the following:
a Tapping application,
a Ballerina application,
a side to side or back and forth shading application, and
a windshield wiper shading application.

27. The method of claim 26 wherein the application process includes both powered and non-powered techniques with a common needle device and a common needle hand support.

28. The method of claim 27 wherein the skin penetration area is a lip area and there is additionally utilized an obovoid or looping application technique and a uniform lining application technique.

29. The method of claim 26 wherein the skin penetration area is an eyeliner skin area and there is additionally utilized a uniform lining application technique.

30. The method of claim 26 wherein the skin penetration area is an eyebrow skin area and there is additionally utilized a graduated hairstroke lining application technique.

31. A holder tip for a needle device wherein said holder tip has a housing defining a reception area for receiving a needle device having a plurality of needles arranged as to have needle tips extending along at least one sloping edge, and wherein said holder tip has at least one sloped edge rim wall that defines an exit opening for the needles and which sloped edge rim slopes in a fashion that conforms to the slope of the sloping edge of the needle device when received in position for use the holder tip.

32. The holder tip of claim 31 wherein said housing is arranged to receive a needle set having a plurality of needle tip defined sloping edges and wherein said sloped edge rim wall has a plurality of sloped edge rim wall sections that conform in slope to respective ones of the needle tip defined sloping edges.
